SEQUENCE LISTING (101 sequences)

SEQ ID NO: 1 - DNA, 50 bp, Artificial Sequence
  source: /note=“Description of Artificial Sequence Synthetic oligonucleotide”
tacgcgcaattcgccttgtcggtgatcttcctttgaacttgggcagtctg

SEQ ID NO: 2 - DNA, 50 bp, Artificial Sequence
  source: /note=“Description of Artificial Sequence Synthetic oligonucleotide”
tggcctggccgtgtcgtctgctttatagtcggtgatcccttgtgttaatt

SEQ ID NO: 3 - DNA, 50 bp, Artificial Sequence
  source: /note=“Description of Artificial Sequence Synthetic oligonucleotide”
gcatacctttcgtatgcctttttgacccgtatttttgccctacccttcgg

SEQ ID NO: 4 - DNA, 50 bp, Artificial Sequence
  source: /note=“Description of Artificial Sequence Synthetic oligonucleotide”
tttttcgctatccaacccttctttccagcctgccaatcagtcggtgatca

SEQ ID NO: 5 - DNA, 50 bp, Artificial Sequence
  source: /note=“Description of Artificial Sequence Synthetic oligonucleotide”
agggcagtcctgtatcttaacattctcctacatccgtaagtcggtgatcc

SEQ ID NO: 6 - DNA, 50 bp, Artificial Sequence
  source: /note=“Description of Artificial Sequence Synthetic oligonucleotide”
gggctaagagtctctattgtcggcagtcgtctaatatttcccgtccaatt

SEQ ID NO: 7 - DNA, 50 bp, Artificial Sequence
  source: /note=“Description of Artificial Sequence Synthetic oligonucleotide”
acttccggtgatttgatttcacttcctgggcagtcaatgtgattctctac

SEQ ID NO: 8 - DNA, 50 bp, Artificial Sequence
  source: /note=“Description of Artificial Sequence Synthetic oligonucleotide”
ggtcagtcgcctttgtcggtgatgtactcgcgcagtcgggttccccttaa

SEQ ID NO: 9 - DNA, 50 bp, Artificial Sequence
  source: /note=“Description of Artificial Sequence Synthetic oligonucleotide”
gggtctgttggtcctagggcagtcgtacttctaattcttgtcccgatgat

SEQ ID NO: 10 - DNA, 50 bp, Artificial Sequence
  source: /note=“Description of Artificial Sequence Synthetic oligonucleotide”
cttgtcggtgatctatagtcggtgatatattttgtcctatggtagtcgat

SEQ ID NO: 11 - DNA, 50 bp, Artificial Sequence
  source: /note=“Description of Artificial Sequence Synthetic oligonucleotide”
gggctcatgggcagtctttttactacctcctatttacgtatcccgctcct

SEQ ID NO: 12 - DNA, 50 bp, Artificial Sequence
  source: /note=“Description of Artificial Sequence Synthetic oligonucleotide”
cacccgcgcatttccccccagtcggtgattcttatatgtacctgttcctc

SEQ ID NO: 13 - DNA, 50 bp, Artificial Sequence
  source: /note=“Description of Artificial Sequence Synthetic oligonucleotide”
gggcacgtccattcgcgtttttgttccgtttctccctttttggattttgc

SEQ ID NO: 14 - DNA, 50 bp, Artificial Sequence
  source: /note=“Description of Artificial Sequence Synthetic oligonucleotide”
cagtcggtgtcactccagcggtcggttcactccacattctcccatctgtc

SEQ ID NO: 15 - DNA, 50 bp, Artificial Sequence
  source: /note=“Description of Artificial Sequence Synthetic oligonucleotide”
ggcagtcaccattctctttgggcagattgtctctcatccatatgtctcct

SEQ ID NO: 16 - DNA, 50 bp, Artificial Sequence
  source: /note=“Description of Artificial Sequence Synthetic oligonucleotide”
ctacctccttagtcggtgattcgatctatgggcctaactgccttctctgt

SEQ ID NO: 17 - DNA, 50 bp, Artificial Sequence
  source: /note=“Description of Artificial Sequence Synthetic oligonucleotide”
gggatgcggggccccgttctttttgtctctcattttgtcactttttttgt

SEQ ID NO: 18 - DNA, 50 bp, Artificial Sequence
  source: /note=“Description of Artificial Sequence Synthetic oligonucleotide”
ggtcagtcccttcggcatgtcgggattccctcttttcgcctcgtttcttt

SEQ ID NO: 19 - DNA, 50 bp, Artificial Sequence
  source: /note=“Description of Artificial Sequence Synthetic oligonucleotide”
ggctgtcgaactttctccctcccaccgcagtcggcccctcatcagtcgta

SEQ ID NO: 20 - DNA, 50 bp, Artificial Sequence; identical to SEQ ID NO: 7
  source: /note=“Description of Artificial Sequence Synthetic oligonucleotide”

SEQ ID NO: 21 - DNA, 50 bp, Artificial Sequence
  source: /note=“Description of Artificial Sequence Synthetic oligonucleotide”
acgtctgtcggtgacctgtaatagtttatgtcggtgatacagctttccct

SEQ ID NO: 22 - DNA, 50 bp, Artificial Sequence
  source: /note=“Description of Artificial Sequence Synthetic oligonucleotide”
ctgtcggtgatcatataacgcagtcggtgtagtttaatcccactccccta

SEQ ID NO: 23 - DNA, 50 bp, Artificial Sequence
  source: /note=“Description of Artificial Sequence Synthetic oligonucleotide”
ggccagtgtcccagtcgtgattgtaatattagattctttgtggcagtcgt

SEQ ID NO: 24 - DNA, 50 bp, Artificial Sequence
  source: /note=“Description of Artificial Sequence Synthetic oligonucleotide”
actcgtcggtgattttagacctttctcggtgatcaacacgtcatgctatt

SEQ ID NO: 25 - DNA, 50 bp, Artificial Sequence
  source: /note=“Description of Artificial Sequence Synthetic oligonucleotide”
gcctcgatatcctcaggagtcggtgtttcattcaatcgtcggtgataaat

SEQ ID NO: 26 - DNA, 50 bp, Artificial Sequence
  source: /note=“Description of Artificial Sequence Synthetic oligonucleotide”
ggtcagtccgtataccgccaatccgaaccgcagtcggtgtccgcttttac

SEQ ID NO: 27 - DNA, 50 bp, Artificial Sequence
  source: /note=“Description of Artificial Sequence Synthetic oligonucleotide”
tcgggttagatgtcggtcccactatatgtcggtgatctaatattgaactt

SEQ ID NO: 28 - DNA, 73 bp, Artificial Sequence
  source: /note=“Description of Artificial Sequence Synthetic oligonucleotide”
tcgtcgtcgcggttcgcgtccgtgcatacctttcgtatgcctttttgacccgtatttttgccctacccttcgg

SEQ ID NO: 29 - DNA, 73 bp, Artificial Sequence; identical to SEQ ID NO: 28
  source: /note=“Description of Artificial Sequence Synthetic oligonucleotide”

SEQ ID NO: 30 - DNA, 73 bp, Artificial Sequence; identical to SEQ ID NO: 28
  source: /note=“Description of Artificial Sequence Synthetic oligonucleotide”

SEQ ID NO: 31 - DNA, 73 bp, Artificial Sequence; identical to SEQ ID NO: 28
  source: /note=“Description of Artificial Sequence Synthetic oligonucleotide”

SEQ ID NO: 32 - DNA, 73 bp, Artificial Sequence
  source: /note=“Description of Artificial Sequence Synthetic oligonucleotide”
tcgtcgtcgcggttcgcgtccgtgggctaagagtctctattgtcggcagtcgtctaatatttcccgtccaatt

SEQ ID NO: 33 - DNA, 73 bp, Artificial Sequence
  source: /note=“Description of Artificial Sequence Synthetic oligonucleotide”
tcgtcgtcgcggttcgcgtccgtagggcagtcctgtatcttaacattctcctacatccgtaagtcggtgatcc

SEQ ID NO: 34 - DNA, 73 bp, Artificial Sequence
  source: /note=“Description of Artificial Sequence Synthetic oligonucleotide”
cttaatcagacattatacaaattgcatacctttcgtatgcctttttgacccgtatttttgccctacccttcgg

SEQ ID NO: 35 - DNA, 73 bp, Artificial Sequence
  source: /note=“Description of Artificial Sequence Synthetic oligonucleotide”
cttaatcagacattatacaaattgggctaagagtctctattgtcggcagtcgtctaatatttcccgtccaatt

SEQ ID NO: 36 - DNA, 73 bp, Artificial Sequence
  source: /note=“Description of Artificial Sequence Synthetic oligonucleotide”
cttaatcagacattatacaaattagggcagtcctgtatcttaacattctcctacatccgtaagtcggtgatcc

SEQ ID NO: 37 - DNA, 71 bp, Artificial Sequence
  source: /note=“Description of Artificial Sequence Synthetic oligonucleotide”
gaattaacaattataacgtttgcatacctttcgtatgcctttttgacccgtatttttgccctacccttcgg

SEQ ID NO: 38 - DNA, 71 bp, Artificial Sequence
  source: /note=“Description of Artificial Sequence Synthetic oligonucleotide”
gaattaacaattataacgtttagggcagtcctgtatcttaacattctcctacatccgtaagtcggtgatcc

SEQ ID NO: 39 - DNA, 69 bp, Artificial Sequence
  source: /note=“Description of Artificial Sequence Synthetic oligonucleotide”
gaattaacaattataacgtgggctaagagtctctattgtcggcagtcgtctaatatttcccgtccaatt

SEQ ID NO: 40 - DNA, 50 bp, Artificial Sequence
  source: /note=“Description of Artificial Sequence Synthetic oligonucleotide”
gattgatctattttccatatcgcgttgagtgtaaagccacgaagggttat

SEQ ID NO: 41 - DNA, 73 bp, Artificial Sequence
  source: /note=“Description of Artificial Sequence Synthetic oligonucleotide”
cggacgcgaaccgcgacgacgatgattgatctattttccatatcgcgttgagtgtaaagccacgaagggttat

SEQ ID NO: 42 - DNA, 73 bp, Artificial Sequence; identical to SEQ ID NO: 41
  source: /note=“Description of Artificial Sequence Synthetic oligonucleotide”

SEQ ID NO: 43 - DNA, 89 bp, Artificial Sequence
  source: /note=“Description of Artificial Sequence Synthetic oligonucleotide”
  modified_base (21)..(69): a, c, t, g, unknown or other
tatccgtctgctctcgctatnnnnnnnnnnnnnnnnnnnnnnnnnnnnnnnnnnnnnnnnnnnnnnnnnacgcacctaatgtcctactg

SEQ ID NO: 44 - DNA, 20 bp, Artificial Sequence
  source: /note=“Description of Artificial Sequence Synthetic primer”
tatccgtctgctctcgctat

SEQ ID NO: 45 - DNA, 20 bp, Artificial Sequence
  source: /note=“Description of Artificial Sequence Synthetic primer”
cagtaggacattaggtgcgt

SEQ ID NO: 46 - DNA, 20 bp, Artificial Sequence
  source: /note=“Description of Artificial Sequence Synthetic oligonucleotide”
atagcgagagcagacggata

SEQ ID NO: 47 - DNA, 20 bp, Artificial Sequence
  source: /note=“Description of Artificial Sequence Synthetic primer”
tatccgtctgctctcgctat

SEQ ID NO: 48 - DNA, 50 bp, Artificial Sequence
  source: /note=“Description of Artificial Sequence Synthetic oligonucleotide”
tttttttttttttttttttttttttttttttttttttttttttttttttt

SEQ ID NO: 49 - DNA, 54 bp, Artificial Sequence
  source: /note=“Description of Artificial Sequence Synthetic oligonucleotide”
ccgcgtccggacacctaatttggttcaagagccgcccgtaatttcaggttctcc

SEQ ID NO: 50 - DNA, 50 bp, Artificial Sequence; identical to SEQ ID NO: 3
  source: /note=“Description of Artificial Sequence Synthetic oligonucleotide”

SEQ ID NO: 51 - DNA, 50 bp, Artificial Sequence
  source: /note=“Description of Artificial Sequence Synthetic oligonucleotide”
gttcttataatcgcctctgcgctatgttcttgctcgccttccatatcgct

SEQ ID NO: 52 - DNA, 50 bp, Artificial Sequence; identical to SEQ ID NO: 51
  source: /note=“Description of Artificial Sequence Synthetic oligonucleotide”

SEQ ID NO: 53 - DNA, 50 bp, Artificial Sequence
  source: /note=“Description of Artificial Sequence Synthetic oligonucleotide”
tctttcgttagcgcttctctcttgcgattccgaccgcatattcacgtctt

SEQ ID NO: 54 - DNA, 80 bp, Artificial Sequence
  source: /note=“Description of Artificial Sequence Synthetic oligonucleotide”
  modified_base (21)..(60): a, c, t, g, unknown or other
tcactatcggtccagacgtannnnnnnnnnnnnnnnnnnnnnnnnnnnnnnnnnnnnnnntattgcgccgaggttcttac

SEQ ID NO: 55 - DNA, 20 bp, Artificial Sequence
  source: /note=“Description of Artificial Sequence Synthetic primer”
tcactatcggtccagacgta

SEQ ID NO: 56 - DNA, 20 bp, Artificial Sequence
  source: /note=“Description of Artificial Sequence Synthetic primer”
tcactatcggtccagacgta

SEQ ID NO: 57 - DNA, 20 bp, Artificial Sequence
  source: /note=“Description of Artificial Sequence Synthetic primer”
gtaagaacctcggcgcaata

SEQ ID NO: 58 - DNA, 20 bp, Artificial Sequence
  source: /note=“Description of Artificial Sequence Synthetic oligonucleotide”
tacgtctggaccgatagtga

SEQ ID NO: 59 - DNA, 40 bp, Artificial Sequence
  source: /note=“Description of Artificial Sequence Synthetic oligonucleotide”
atcgtataagggctgcttaggattgcgataatacggtcaa

SEQ ID NO: 60 - DNA, 40 bp, Artificial Sequence
  source: /note=“Description of Artificial Sequence Synthetic oligonucleotide”
catttcatagggctgcttaggattgcgaaggtaatgccag

SEQ ID NO: 61 - DNA, 40 bp, Artificial Sequence
  source: /note=“Description of Artificial Sequence Synthetic oligonucleotide”
cccttaccccttttaggtctgcttaggattgcgaaaaaag

SEQ ID NO: 62 - DNA, 40 bp, Artificial Sequence
  source: /note=“Description of Artificial Sequence Synthetic oligonucleotide”
ttgtaaggactgcttaggattgcgaaaacaatattcgtat

SEQ ID NO: 63 - DNA, 31 bp, Artificial Sequence
  source: /note=“Description of Artificial Sequence Synthetic oligonucleotide”
cttttaggtctgcttaggattgcgaaaaaag

SEQ ID NO: 64 - DNA, 40 bp, Artificial Sequence
  source: /note=“Description of Artificial Sequence Synthetic oligonucleotide”
tccatgggtctgctctaggattgcgttcatggtctccccg

SEQ ID NO: 65 - DNA, 40 bp, Artificial Sequence
  source: /note=“Description of Artificial Sequence Synthetic oligonucleotide”
aattacaaccttggattgcaaagggctgctgtgttgttta

SEQ ID NO: 66 - DNA, 40 bp, Artificial Sequence
  source: /note=“Description of Artificial Sequence Synthetic oligonucleotide”
atcggagctgttccttgataccgattcaaaaagttcgtac

SEQ ID NO: 67 - DNA, 40 bp, Artificial Sequence
  source: /note=“Description of Artificial Sequence Synthetic oligonucleotide”
aatttgtagggactgctcaggattgcggatacaaattaat

SEQ ID NO: 68 - DNA, 40 bp, Artificial Sequence
  source: /note=“Description of Artificial Sequence Synthetic oligonucleotide”
agacattggggactgctcgggattgcgaatctatgtctcc

SEQ ID NO: 69 - DNA, 40 bp, Artificial Sequence
  source: /note=“Description of Artificial Sequence Synthetic oligonucleotide”
cccttttttaactaggtctgcttaggattgcgaatgttaa

SEQ ID NO: 70 - DNA, 40 bp, Artificial Sequence
  source: /note=“Description of Artificial Sequence Synthetic oligonucleotide”
acctcaaaagcgcgggctgctcaaaggattgcgtagcttt

SEQ ID NO: 71 - DNA, 40 bp, Artificial Sequence
  source: /note=“Description of Artificial Sequence Synthetic oligonucleotide”
gggggttaagggctgcttaggattgcgataatacggtcaa

SEQ ID NO: 72 - DNA, 40 bp, Artificial Sequence
  source: /note=“Description of Artificial Sequence Synthetic oligonucleotide”
aacatataactgctcaataatatagataaaatactcacaa

SEQ ID NO: 73 - DNA, 40 bp, Artificial Sequence
  source: /note=“Description of Artificial Sequence Synthetic oligonucleotide”
ctctacctgactgtaacctctcgctcccccccattcgcgc

SEQ ID NO: 74 - DNA, 40 bp, Artificial Sequence
  source: /note=“Description of Artificial Sequence Synthetic oligonucleotide”
ttgtccctctacgccgccctttactaccactcctgcgatt

SEQ ID NO: 75 - DNA, 40 bp, Artificial Sequence
  source: /note=“Description of Artificial Sequence Synthetic oligonucleotide”
tccagcacaccgaccgcccctctacattaccccctggact

SEQ ID NO: 76 - DNA, 40 bp, Artificial Sequence
  source: /note=“Description of Artificial Sequence Synthetic oligonucleotide”
cccctccattcccccgcctcgtccaccctactccttagtc

SEQ ID NO: 77 - DNA, 40 bp, Artificial Sequence
  source: /note=“Description of Artificial Sequence Synthetic oligonucleotide”
catcgacgcccacacaccacttcccgttcccctgcatcat

SEQ ID NO: 78 - DNA, 63 bp, Artificial Sequence
  source: /note=“Description of Artificial Sequence Synthetic oligonucleotide”
cggacgcgaaccgcgacgacgatagcaatcatatggctgtgctcatttaataagcaagctggg

SEQ ID NO: 79 - DNA, 63 bp, Artificial Sequence
  source: /note=“Description of Artificial Sequence Synthetic oligonucleotide”
cggacgcgaaccgcgacgacgataaactctatcgtccagagagaatgtctgcctactgatttg

SEQ ID NO: 80 - DNA, 63 bp, Artificial Sequence
  source: /note=“Description of Artificial Sequence Synthetic oligonucleotide”
tcgtcgtcgcggttcgcgtccgtatcgtataagggctgcttaggattgcgataatacggtcaa

SEQ ID NO: 81 - DNA, 25 bp, Artificial Sequence
  source: /note=“Description of Artificial Sequence Synthetic oligonucleotide”
agggctgcttaggattgcgataata

SEQ ID NO: 82 - DNA, 25 bp, Artificial Sequence
  source: /note=“Description of Artificial Sequence Synthetic oligonucleotide”
agggctgcttaggattgcgaaggta

SEQ ID NO: 83 - DNA, 26 bp, Artificial Sequence
  source: /note=“Description of Artificial Sequence Synthetic oligonucleotide”
gggtctgctctaggattgcgttcatg

SEQ ID NO: 84 - DNA, 26 bp, Artificial Sequence
  source: /note=“Description of Artificial Sequence Synthetic oligonucleotide”
ggagctgttccttgataccgattcaa

SEQ ID NO: 85 - DNA, 25 bp, Artificial Sequence
  source: /note=“Description of Artificial Sequence Synthetic oligonucleotide”
gggactgctcaggattgcggataca

SEQ ID NO: 86 - DNA, 25 bp, Artificial Sequence
  source: /note=“Description of Artificial Sequence Synthetic oligonucleotide”
aggtctgcttaggattgcgaaaaaa

SEQ ID NO: 87 - DNA, 25 bp, Artificial Sequence
  source: /note=“Description of Artificial Sequence Synthetic oligonucleotide”
gggactgctcgggattgcgaatcta

SEQ ID NO: 88 - DNA, 25 bp, Artificial Sequence
  source: /note=“Description of Artificial Sequence Synthetic oligonucleotide”
aggtctgcttaggattgcgaatgtt

SEQ ID NO: 89 - DNA, 25 bp, Artificial Sequence
  source: /note=“Description of Artificial Sequence Synthetic oligonucleotide”
aggactgcttaggattgcgaaaaca

SEQ ID NO: 90 - DNA, 27 bp, Artificial Sequence
  source: /note=“Description of Artificial Sequence Synthetic oligonucleotide”
cgggctgctcaaaggattgcgtagctt

SEQ ID NO: 91 - DNA, 26 bp, Artificial Sequence
  source: /note=“Description of Artificial Sequence Synthetic oligonucleotide”
ataactgctcaataatatagataaaa

SEQ ID NO: 92 - DNA, 27 bp, Artificial Sequence
  source: /note=“Description of Artificial Sequence Synthetic oligonucleotide”
taagggctgcttaggattgcgataata

SEQ ID NO: 93 - DNA, 27 bp, Artificial Sequence
  source: /note=“Description of Artificial Sequence Synthetic oligonucleotide”
atagggctgcttaggattgcgaaggta

SEQ ID NO: 94 - DNA, 27 bp, Artificial Sequence
  source: /note=“Description of Artificial Sequence Synthetic oligonucleotide”
ttaggtctgcttaggattgcgaaaaaa

SEQ ID NO: 95 - DNA, 27 bp, Artificial Sequence
  source: /note=“Description of Artificial Sequence Synthetic oligonucleotide”
taaggactgcttaggattgcgaaaaca

SEQ ID NO: 96 - DNA, 27 bp, Artificial Sequence
  source: /note=“Description of Artificial Sequence Synthetic oligonucleotide”
tggggactgctcgggattgcgaatcta

SEQ ID NO: 97 - DNA, 40 bp, Artificial Sequence; identical to SEQ ID NO: 59
  source: /note=“Description of Artificial Sequence Synthetic oligonucleotide”

SEQ ID NO: 98 - DNA, 40 bp, Artificial Sequence; identical to SEQ ID NO: 60
  source: /note=“Description of Artificial Sequence Synthetic oligonucleotide”

SEQ ID NO: 99 - DNA, 40 bp, Artificial Sequence; identical to SEQ ID NO: 61
  source: /note=“Description of Artificial Sequence Synthetic oligonucleotide”

SEQ ID NO: 100 - DNA, 31 bp, Artificial Sequence; identical to SEQ ID NO: 63
  source: /note=“Description of Artificial Sequence Synthetic oligonucleotide”

SEQ ID NO: 101 - DNA, 40 bp, Artificial Sequence; identical to SEQ ID NO: 62
  source: /note=“Description of Artificial Sequence Synthetic oligonucleotide”

(19) United States
(12) Patent Application Publication
(10) Pub. No.: US 2023/0193286 A1
(43) Pub. Date: Jun. 22, 2023

(54) CYTOTOXIC T-LYMPHOCYTE BINDING APTAMERS

(71) Applicant: Aummune Ltd., Tel Aviv-Yafo (IL)

(72) Inventors: Guy NEEV, Bnei Dror (IL); Irit Carmi-Levy, Tel Aviv (IL); Yaniv Amir, Tel Aviv (IL); Erez Lavi, Bat Yam (IL); Nir Skalka, Rehovot (IL); Neta Zilony-Hanin, Tel Aviv (IL); Zohar Pode, Tel Aviv (IL)

(21) Appl. No.: 17/926,071

(22) PCT Filed: May 19, 2021
(86) PCT No.: PCT/IB2021/000345

§ 371 Date: Nov. 17, 2022

Related U.S. Application Data
(60) Provisional application No. 63/027,631, filed on May 20, 2020.

(51) Int. Cl. C12N 15/115; A61P 35/00; A61K 45/06

(52) U.S. Cl. CPC C12N 15/115; A61P 35/00; A61K 45/06; C12N 2310/16; C12N 2310/351; C12N 2310/531; C12N 2320/13; C12N 2310/51; C12N 2310/322; C12N 2310/321; C12N 2310/3231; C12N 2310/315; C12N 2310/3515; C12N 2320/31

(57) ABSTRACT

Provided herein are aptamers that target cytotoxic T-lymphocyte and methods of use thereof.

### RELATED APPLICATIONS

This application claims the benefit of priority to U.S. Provisional Patent Application Ser. No. 63/027,631, filed May 20, 2020, which is hereby incorporated by reference in its entirety.

### BACKGROUND

Bispecific molecules harnessing and redirecting the cytotoxicity of effector T-cells towards tumor cells are a promising therapeutic agent. Naturally occurring IgG antibodies do not have the functionality to directly engage cytotoxic T lymphocytes (CTL). Over the past three decades, a myriad of T-bispecific antibodies have been developed. Although the molecular details differ considerably, they are all grounded on the basic design of combining tumor antigen-binding specificity and T cell-binding specificity into one molecule, with or without an Fc region. To date, only a single T-bispecific antibody, blinatumomab, has been approved for clinical use in humans, as compared to more than 25 IgG other based antibody drugs. The lag is largely attributed to the difficulties in protein engineering during the manufacture of these antibodies and the uncertain clinical toxicities of these novel constructs (Wu and Cheung (2018) Pharmacol. Ther. 182:161-175).

Aptamers are single stranded oligonucleotides which bind tightly and specifically to a variety of targets, including proteins, sugars, and small organic compounds. There is increasing interest in using aptamers for the development of both therapeutics and diagnostics.

Although aptamers recognize and bind targets of interest like antibodies, they have a number of advantages, such as shorter generation time, lower costs of manufacturing, low batch-to-batch variability, higher modifiability, better thermal stability and low immunogenicity (Zhang, Lai, and Juhas (2019) Molecules 24: pii: E941. doi: 10.3390/molecules24050941).

Thus, aptamers that are capable of targeting T cells would have great potential for use as anti-cancer therapeutics.

### SUMMARY

In certain aspects, provided herein are aptamers that bind to T cells (e.g., CD8+ T cells) and/or that induce T cell stimulation and/or T cell-mediated cytotoxicity. In some aspects, provided herein are pharmaceutical compositions comprising such aptamers, methods of using such aptamers to treat cancer and/or to kill cancer cells and methods of making such aptamers.

In certain aspects, provided herein are aptamers comprising a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 1-39, 59-77 or 80 (Tables 11, 16, 17, and 18). In certain embodiments, the aptamers comprise at least 20 (e.g., at least 25, at least 30, at least 35, at least 40, at least 41, at least 42, at least 43, at least 44, at least 45, at least 46, at least 47, at least 48, at least 49, or at least 50) consecutive nucleotides of any one of SEQ ID NO: 1-27 or 59-77. In certain embodiments, the aptamers comprise at least 40 (e.g., at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, or at least 73) consecutive nucleotides of any one of SEQ ID NO: 28-39 or 80. In some embodiments, the aptamers comprise a nucleic acid sequence of any one of SEQ ID NOs: 1-39, 59-77 or 80 (e.g., any one of SEQ ID NOs: 3, 5, 6, 28, 59, 80, and 29). In some embodiments, the aptamers provided herein have a sequence consisting essentially of any one of SEQ ID NOs: 1-39, 59-77 or 80 (e.g., any one of SEQ ID NOs: 3, 5, 6, 28, 59, 80, and 29). In certain embodiments, the aptamers provided herein have a sequence consisting of any one of SEQ ID NO: 1-39, 59-77 or 80 (e.g., any one of SEQ ID NOs: 3, 5, 6, 28, 59, 80, and 29).

In certain embodiments, the aptamers provided herein are no more than 100 nucleotides in length (e.g., no more than 90 nucleotides in length, no more than 85 nucleotides in length, no more than 80 nucleotides in length, no more than 75 nucleotides in length, no more than 73 nucleotides in length, no more than 70 nucleotides in length, no more than 65 nucleotides in length, no more than 60 nucleotides in length, no more than 59 nucleotides in length, no more than 58 nucleotides in length, no more than 57 nucleotides in length, no more than 56 nucleotides in length, no more than 55 nucleotides in length, no more than 54 nucleotides in length, no more than 53 nucleotides in length, no more than 52 nucleotides in length, no more than 51 nucleotides in length, or no more than 50 nucleotides in length).

In some embodiments, the aptamers provided herein are able to bind to a T cell (e.g., a CD8+ cytotoxic T cell). In some embodiments, the aptamers provided herein bind to a T cell antigen selected from Notch 2 and other Notch family members, KCNK17, CD3, CD28, 4-1BB, CTLA-4, ICOS, CD40L, PD-1, OX40, LFA-1, CD27 PARP16, IGSF9, SLC15A3 and WRB. In some embodiments, the aptamers provided herein bind to the T cell surface protein CD3 (e.g. CD3 epsilon chain, CD3e). In some embodiments, the aptamers are able to induce T cell-mediated cytotoxicity. In some embodiments, the aptamers are able to induce (a) cytokine secretion; and/or (b) T cell activation. In some embodiments, the aptamers are able to induce cell death of a cancer cell (e.g., a human cancer cell) through T cell-mediated cytotoxicity. In some embodiments, the cancer cell is a patient-derived cancer cell. In some embodiments, the cancer cell is a solid tumor cell. In certain embodiments, the cancer cell is a colorectal carcinoma cell. In some embodiments, the cancer cell is a breast cancer cell. In some embodiments, the aptamers induce cell death of a cancer cell in vitro. In certain embodiments, the aptamers induce cell death of a cancer cell in vivo (e.g., in a human and/or an animal model).

In some embodiments, the aptamers provided herein comprise one or more chemical modifications. In some embodiments, the aptamers are chemically modified with poly-ethylene glycol (PEG) (e.g., attached to the 5′ end of the aptamer). In some embodiment, the aptamers comprise a 5′ end cap. In certain embodiments, the aptamers comprise a 3′ end cap (e.g., is an inverted thymidine, biotin). In some embodiments, the aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 3031, 32, 33, 34, 35, 36, 37, 38, 39, 40, 41, 42, 43, 44, 45, 46, 47, 48, 49, 50, 51, 52, 53, or 54) 2′ sugar substitutions (e.g. a 2′-fluoro, a 2′-amino, or a 2′-O-methyl substitution). In certain embodiments, the aptamers comprise locked nucleic acid (LNA), unlocked nucleic acid (UNA) and/or 2′deozy-2′fluoro-D-arabinonucleic acid (2′-F ANA) sugars in their backbone. In certain embodiments, the aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) methylphosphonate internucleotide bonds and/or a phosphorothioate (PS) internucleotide bonds. In certain embodiments, the aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 3031, 32, 33, 34, 35, 36, 37, 38, 39, 40, 41, 42, 43, 44, 45, 46, 47, 48, 49, 50, 51, 52, 53, or 54) triazole internucleotide bonds. In certain embodiments, the aptamers are modified with a cholesterol or a dialkyl lipid (e.g., on their 5′ end). In some embodiments, the aptamers comprise one or more modified bases.

In certain embodiments, the aptamers provided herein are DNA aptamers (e.g., D-DNA aptamers or enantiomer L-DNA aptamers). In some embodiments, the aptamers provided herein are RNA aptamers (e.g., D-RNA aptamers or enantiomer L-RNA aptamers). In some embodiments, the aptamers comprise a mixture of DNA and RNA.

In certain aspects, provided herein are aptamer conjugates comprising an aptamer provided herein linked to a cancer cell-binding moiety (e.g., a small molecule, another aptamer, a polypeptide, a nucleic acid, a protein, and/or an antibody). In some embodiments, the aptamer is covalently linked to the cancer cell-binding moiety. In some embodiments, the aptamer is non-covalently linked to the cancer cell-binding moiety. In some embodiments, the aptamer is directly linked to the cancer cell-binding moiety. In some embodiments, the aptamer is linked to the cancer cell-binding moiety via a linker. In some embodiments, the cancer-cell binding moiety binds to an antigen expressed on a cancer cell. In some embodiments, the antigen expressed on the cancer cell is selected from Prostate-specific antigen (PSA), Prostate Membrane Antigen (PSMA), Cancer antigen 15-3 (CA-15-3), Carcinoembryonic antigen (CEA), Cancer antigen 125 (CA-125), Alpha-fetoprotein (AFP), NY-ESO-1, MAGEA-A3, WT1, hTERT, Tyrosinase, gp100, MART-1, melanA, B catenin, CDC27, HSP70-2-m, HLA-A2-R170J, AFP, EBV-EBNA, HPV16-E7, MUC-1, HER-2/neu, Mammaglobin-A or MHC-TAA peptide complexes. In some embodiments, the cancer-cell binding moiety induces cell death (e.g., apoptosis) when contacted to a cancer cell (e.g., a human cancer cell). In some embodiments, the cancer cell is a patient-derived cancer cell. In some embodiments, the cancer cell is a solid tumor cell. In certain embodiments, the cancer cell is a colorectal carcinoma cell. In some embodiments, the cancer cell is a breast cancer cell. In some embodiments, the cancer-cell binding moiety induces cell death when contacted to a cancer cell in vitro. In certain embodiments, the cancer-cell binding moiety induces cell death when contacted to a cancer cell in vivo (e.g., in a human and/or an animal model).

In certain aspects, provided herein are pharmaceutical compositions comprising an aptamer (e.g., a therapeutically effective amount of an aptamer) provided herein. In certain aspects, provided herein are pharmaceutical compositions comprising an aptamer conjugate (e.g., a therapeutically effective amount of an aptamer conjugate) provided herein. In some embodiments, the pharmaceutical compositions provided herein further comprise a pharmaceutically acceptable carrier. In some embodiments, the pharmaceutical compositions provided herein are formulated for parenteral administration.

In certain embodiments, the pharmaceutical compositions provided herein are for use in treating cancer. In some embodiments, the cancer is a solid tumor. In certain embodiments, the cancer is a colorectal carcinoma. In some embodiments, the cancer is a breast cancer.

In certain aspects, provided herein is a method of treating cancer in a subject, the method comprising administering to the subject an aptamer (e.g., a therapeutically effective amount of an aptamer) or a pharmaceutical composition provided herein. In certain aspects, provided herein is a method of treating cancer in a subject, the method comprising administering to the subject an aptamer conjugate (e.g., a therapeutically effective amount of an aptamer conjugate) or a pharmaceutical composition provided herein. In some embodiments, the administration is parenteral administration (e.g., subcutaneous administration). The administration may be an intratumoral injection or a peritumoral injection.

In some embodiments, the cancer is a solid tumor. In certain embodiments, the cancer is a colorectal carcinoma. In some embodiments, the cancer is a breast cancer, head and neck squamous cell carcinoma, adenoid cystic carcinoma, bladder cancer, pancreatic cancer, hepatocellular carcinoma, melanoma, or merkel cell carcinoma. In certain embodiments, the subject is a subject who has received chemotherapy. In certain embodiments, the subject is a subject who has had a tumor surgically removed (e.g., who has had a breast cancer tumor surgically removed).

In some embodiments, the therapeutic methods provided herein further comprise administering to the subject an additional cancer therapy. In some embodiments, the additional cancer therapy comprises chemotherapy. In certain embodiments, the additional cancer therapy comprises radiation therapy. In some embodiments, the additional cancer therapy comprises surgical removal of a tumor. In certain embodiments, the additional cancer therapy comprises administration of an immune checkpoint inhibitor, e.g., an anti-PD-1 antibody, an anti-PD-L1 antibody, an anti-PD-L2 antibody, or an anti-CTLA4 antibody) to the subject.

In certain aspects, provided herein is a method of killing a cancer cell, the method comprising contacting the cancer cell with an aptamer or an aptamer conjugate provided herein. In some embodiments, the cancer cell is killed by apoptosis. In some embodiments, the cancer cell is a solid tumor cell. In certain embodiments, the cancer cell is a colorectal carcinoma cell. In some embodiments, the cancer cell is a breast cancer cell. In some embodiments, the cancer cell is killed when contacted with the cancer cell in vitro. In certain embodiments, the cancer cell is killed when contacted with the cancer cell in vivo (e.g., in a human and/or an animal model).

In certain aspects, provided herein is a method of making an aptamer. In some embodiments, the method comprises synthesizing (e.g., chemically synthesizing) a nucleic acid comprising a sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 1-39, 59-77 or 80 (e.g., any one of SEQ ID NOs: 3, 5, 6, 28, 59, 80, and 29). In certain embodiments, the method comprises synthesizing a nucleic acid comprising a sequence that comprises at least 20 (e.g., at least 25, at least 30, at least 35, at least 40, at least 41, at least 42, at least 43, at least 44, at least 45, at least 46, at least 47, at least 48, at least 49, or at least 50) consecutive nucleotides of any one of SEQ ID NO: 1-27 or 59-77 (e.g., any one of SEQ ID NOs: 3, 5, 59, and 6). In certain embodiments, the aptamers comprise at least 40 (e.g., at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, or at least 73) consecutive nucleotides of any one of SEQ ID NO: 28-39 or 80 (e.g., any one of SEQ ID NOs: 28, 80, and 29). In certain embodiments, the method comprises synthesizing a nucleic acid comprising a sequence of any one of SEQ ID NOs: 1-39, 59-77 or 80 (e.g., any one of SEQ ID NOs: 3, 5, 6, 28, 59, 80, and 29). In some embodiments, the method comprises synthesizing a nucleic acid having a sequence consisting essentially of SEQ ID NOs: 1-39, 59-77 or 80 (e.g., any one of SEQ ID NOs: 3, 5, 6, 28, 59, 80, and 29). In certain embodiments, the method comprises synthesizing a nucleic acid having a sequence consisting of SEQ ID NO: 1-39, 59-77 or 80 (e.g., any one of SEQ ID NOs: 3, 5, 6, 28, 59, 80, and 29).

In certain aspects, provided herein is a method of treating an autoimmune disorder in a subject, the method comprising administering to the subject an aptamer (e.g., a therapeutically effective amount of an aptamer) or a pharmaceutical composition provided herein.

In certain aspects, provided herein is a method of treating an inflammatory disease in a subject, the method comprising administering to the subject an aptamer (e.g., a therapeutically effective amount of an aptamer) or a pharmaceutical composition provided herein.

In certain aspects, provided herein is a method of inhibiting transplant rejection in a subject, the method comprising administering to the subject an aptamer (e.g., a therapeutically effective amount of an aptamer) or a pharmaceutical composition provided herein.

### BRIEF DESCRIPTION OF FIGURES

FIG. 1 shows the scheme of CTL Binding Cell-SELEX process. Rounds 1 and 2 were done using cells of donor #1 (labelled in blue). Rounds 3, 4, and 6 were done using cells from donor #2 (labelled in cyan). Negative selection was done after rounds 3 and 4 with CD8-negative cells of donor #1 and donor #2, respectively. The final round, round 7, was repeated three times: one time at “normal” conditions (i.e., 3× wash & short incubation time), one time with long incubation time before the last wash (“long wash”) and finally with twice the number of washes (“6× wash”). Round 7 was done using cells from donor 43.

FIGS. 2A and 2B show the binding SELEX comparative assay. Isolated CD8 T cells were incubated either with the random library 2.6, or with one of the binding SELEX outcome of rounds 4, 6 or 7 tagged with Cy-5 for 1 hour at 37° C. Cy-5 fluorescence intensity was assayed using flow cytometry. FIG. 2A shows the histograms of Cy-5 fluorescence intensity of each round. FIG. 2B shows the fold change of each round over the initial library random 2.6 library.

FIGS. 3A-3D show next generation sequencing (NGS) analysis results. FIG. 3A shows relative abundance of individual sequences in the different rounds sequenced (R2, R5, R6 and R7). Top 100 most abundant sequences of the final enriched library R7 are displayed in grey. Top 10 most abundant sequences are displayed in color. FIG. 3B shows R7 bound-to-unbound ratio of individual sequences identified following the “long wash” stringency plotted against relative abundance in R7. Selected sequences are shown in color. FIG. 3C shows R7 bound-to-unbound ratio of individual sequences in the 6× wash stringency plotted against relative abundance in R7. Selected sequences are shown in color. FIG. 3D shows R7 bound-to-unbound ratio of individual sequences in the “long wash” stringency plotted against R7 bound-to-unbound ratio of individual sequences in the 6× wash stringency.

FIG. 4 show the initial screen of putative aptamers for binding to CD8 cells via flow cytometry. Isolated T cell fluorescence was measured after each wash cycle for a total of three washes. Results were normalized to the “random” aptamer in each wash. N=1 or 2.

FIG. 5 depicts promising CD8 cell binding candidate, CTL3, predicted structure by NUPACK (Zadeh et al. (2011) J. Comput. Chem. 32:170-173).

FIG. 6 shows that CTL3 binds PBMCs. CTL3 aptamer exhibited significantly higher binding affinity to total PBMCs compared with control aptamers. Cy-5 labelled CTL3, random aptamer sequence (RND) and Poly T aptamers each at 250 nM, were tested for their binding post 1 hour (hr) incubation at 4° C. Unstained cells represented cells without aptamer. N=3.

FIGS. 7A-7D show CTL3 binding to different PBMC sub-populations. CTL3 bound to lymphocytes while no significant binding to monocytes was observed (FIGS. 7A and 7B). CTL3 bound to CD8 positive and negative cells equally (FIGS. 7C and 7D). Cy-5 labelled CTL3, RND and Poly T aptamers each at 250 nM, were tested for their binding following 1 hr incubation at 4° C. Unstained cells represented cells without aptamer. N=3.

FIGS. 8A and 8B show CTL3 binding compared with the scrambled sequence. CTL3 aptamer exhibited significantly binding affinity to PBMC (FIG. 8A) and to CD8 T cells and (FIG. 8B) compared with control scrambled (SCR) aptamer. Cy-5 labelled CTL3 and CTL3 SCR aptamers each at 250 nM, were tested for their binding following 1 hr incubation at 4° C. Unstained cells represent cells without aptamer. N=3.

FIG. 9 shows that CTL3 bound to isolated CD8 T cells. Cy-5 labelled CTL3, RND and Poly T aptamers each at 250 nM, were tested for their binding to isolated CD8 cells following 1 hr incubation at 4° C. CTL3 Unstained cells represented cells without aptamer.

FIGS. 10A and 10B show that CTL3 bound to activated and expended Pan-T cells. CTL3, RND and Poly T aptamers, were tested for their binding to activated and expanded Pan-T cells at day 11 post-initial activation. CTL3 bound both CD8 positive (FIG. 10A) and negative cells (FIG. 10B) as compared with control aptamers. Cy-5 labelled CTL3, RND and Poly T aptamers each at 250 nM, were tested after 1 hr incubation at 4° C. Unstained cells represented cells without aptamer. N=1.

FIG. 11 shows Integral Molecular's Membrane Proteome Array (MPA) description. MPA is a high-throughput cell-based platform for identifying the membrane protein targets of ligands. Membrane proteins were expressed in human cells on 384-well microplates, and ligand binding was detected by flow cytometry, allowing sensitive detection of both specific and off-target binding.

FIG. 12 shows the membrane protein array screening with CTL3.

FIG. 13 shows target hit validation for CTL3 aptamer by sequential dilution

FIG. 14 shows a schematic of thermofluorimetric analysis (TFA) of aptamer-protein binding. Intercalator fluorescence is low in the melted, free state (left) and high in the folded aptamer or protein bound state (middle, right). Protein binding adds stability, increasing aptamer melting temperature (i.e., Tₘ,bound>Tₘ,unbound). FIG. 14 is adapted from Hu, Kim and Easley (2016) HHS Public Access. 7:7358-7362.

FIG. 15 shows quantitative protein detection with TFA at 100 nM CTL3. Increasing Notch2 concentration (green) and increasing CD160 concentrations (purple) as control. total fluorescence (left) and fluorescent curve derivative (right).

FIG. 16 shows assessment sequences binding to recombinant Notch2. CTL3 and two scrambled DNA sequences were assessed for their binding to recombinant Notch2 FIGS. 17A-17C show Quantitative Protein Binding Detection with TFA. Tₘ profile curves were generated using 100 nM of CS with increasing concentrations of either human recombinant Notch2 (green, upper), mouse recombinant Notch2 (purple, middle), and rat recombinant Notch2 (orange, bottom).

FIGS. 18A and 18B show the scheme of CD3ε binding SELEX process.

FIGS. 19A and 19B show the binding SELEX comparative assay. Binding assay was performed on target protein CD3ε-beads complex (black) or control protein IgG1 (gray) with initial random library (Rnd Lib) and library enriched pools from Rounds 3(R3), 6(R6), 9(R9), and 11(R11). Post incubation and wash the library DNA was eluted and concentration in the supernatant was evaluated via real-time-PCR. The standard curve was performed with a random library (top). Binding of Cy5 fluorescently labeled libraries to Jurkat T cell line and to Pan B cells was demonstrated by flow cytometry (FIG. 19B). Dot plots and histogram graphs are shown. Flow data quantification of Cy5 median fluorescence intensity (MFI) are shown.

FIGS. 20A-20C show next generation sequencing (NGS) analysis results. FIG. 20A shows analysis of single aptamer sequences from 8^{th}, 9^{th}, 10^{th}, and 11^{th} SELEX rounds enriched libraries on dot plot where the X-axis represents mean P-negative and the Y-axis represents mean P-positive. The diagonal line represents the threshold between specific-binder aptamers and low, nonspecific, binding aptamer sequences. Top 5 candidates selected for further examination are indicated with their names. FIG. 20B shows sequences LOGO display of the shared motif (using GLAM2 software) of top 14 specific-binder aptamers (upper) and top 4 selected aptamers (lower). FIG. 20C shows secondary structural analysis (mfold) of the 5 selected candidates. Motif nucleotides location are marked with a red asterisk.

FIG. 21 shows aptamer sequences binding to target protein by HPLC. Folded and Cy5-labelled aptamer candidates were assayed for recombinant Human CD3ε (hCD3ε) binding. Aptamers were incubated for 1 hr at 37° C. with hCD3e or with the negative control IgG1. PolyT was used as a negative control sequence.

FIGS. 22A-22C show CS6 binding to T cells as demonstrated via flow cytometry. Jurkat cells and Kasumi-1 cells were incubate with CpG′-Cy5 labelled CS6, CS7 and CS8c, and analyzed by flow cytometry (FIG. 22A). Jurkat cells and Daudi cells were incubate with CpG′-Cy5 labelled CS6, CS7 and CS8c and analyzed by flow cytometry. MFI quantification is indicated below (FIG. 22B). Isolated pan T cells and pan B cells were incubated with CpG′-Cy5 labeled CS6 and analyzed by flow cytometry. Representation of dot plots with Cy5 (X-axis)/SSC (Y-axis) of T cells and B cells as well as MFI quantification are presented (FIG. 22C).

FIG. 23 shows CS6 effective concentration. Jurkat cells were incubated with serially-diluted concentrations of CpG′-Cy5 labelled CS6 and analyzed by flow cytometry to determine compound's EC₅₀.

FIG. 24 shows binding of CS6 either to the target protein hCD3ε (top) or to a non-specific IgG control protein (bottom) by SPR sensogram.

FIG. 25 shows that bispecific aptamer acts as a T cell engager and stimulates CD69 elevation.

FIG. 26 provides a schematic representation of an exemplary use of T cell engager aptamer as an aptamer conjugate. In this example, the T cell-binding aptamer is linked to a cancer-targeting, second aptamer, to yield a bispecific aptamer entity. Depicted are the three different domains of the therapeutic agent.

FIGS. 27A-27D show three modes-of-actions (MoAs) in solid tumors for an intratumorally administered bispecific personalized aptamer (FIGS. 27A-27C) and its downstream systemic effect (FIG. 27D).

FIG. 28 shows that bispecific personalized aptamer induces tumor cell death in vitro.

FIGS. 29A and 29B show in vivo efficacy of the exemplary bispecific T cell engager aptamer, comprised of CTL3 aptamer (SEQ ID NO 28) hybridized to HCT116, colon carcinoma cell line-targeting aptamer sequence (named VS12). Female NSG mice were implanted SC with HCT-116 tumor cells admixed with human PBMC followed by a treatment with 100 mg/kg T cell engager bispecific personalized aptamers for a total of 10 doses administered SC. HCT116 tumor volume was monitored during the 22 days of the study for CTL3∥VS12 treatment, PolyT∥PolyT (non-specific DNA aptamer), Vehicle and Untreated mice groups (FIG. 29A). Tumors were weighted at the end of the in-life phase (FIG. 29B). Statistical T-test was implemented. ** indicates significant difference (p≤0.005) and *** ((p≤0.001).

FIG. 30 depicts Kaplan-Meier survival analysis of CTL3∥VS12 treated Mice.

FIGS. 31A and 31B show in vivo efficacy of the exemplary bispecific T cell engager aptamer, comprised of CS6 aptamer (SEQ ID NO: 59) hybridized to HCT116, colon carcinoma cell line-targeting aptamer sequence (named VS12; SEQ ID NO: 42). Female NSG mice were implanted SC with HCT-116 tumor cells admixed with human PBMC followed by a treatment with T cell engager bispecific personalized aptamers for a total of 10 doses administered SC. HCT116 tumor volume was monitored for CS6-VS12 treatment, PolyT-PolyT (non-specific DNA aptamer) and Vehicle mice groups (FIG. 31A). Individual mice growth curves are depicted in FIG. 31B. *** indicates significant difference ((p≤0.001).

FIG. 32 depicts Kaplan-Meier survival analysis of treated Mice. ** indicates significant difference ((p 0.01).

FIGS. 33A and 33B show in vivo efficacy of the exemplary bispecific T cell engager aptamer, comprised of CS6 aptamer (SEQ ID NO: 59) hybridized to 4T1, mammary carcinoma cell line-targeting aptamer sequence (named VS32; SEQ ID NO: 79). Female Balb/c mice were implanted SC with 4T1 tumor cells on both flanks of the mouse. Once the primary tumor has reached a size of 50 mm³, a treatment with T cell engager bispecific personalized aptamers commenced using intratumoral route of administration. Primary and secondary tumor volumes were monitored for CS6-VS12 treatment with or without combination with anti-PD1.

### DETAILED DESCRIPTION

### General

In certain aspects, provided herein are aptamers that selectively bind to T cells (e.g., CD8+ T cells) and/or selectively induces T cell stimulation and/or T cell-mediated cytotoxicity. In some aspects, provided herein are pharmaceutical compositions comprising such aptamers, methods of using such aptamers to treat cancer and/or to kill cancer cells and methods of making such aptamers.

### Definitions

For convenience, certain terms employed in the specification, examples, and appended claims are collected here.

The articles “a” and “an” are used herein to refer to one or to more than one (e.g., to at least one) of the grammatical object of the article. By way of example, “an element” means one element or more than one element.

As used herein, the term “aptamer” refers to a short (e.g., less than 200 bases), single stranded nucleic acid molecule (ssDNA and/or ssRNA) able to specifically bind to a target molecule, e.g., a protein or peptide, or to a topographic feature on a target cell.

The term “binding” or “interacting” refers to an association, which may be a stable association, between two molecules, e.g., between an aptamer and target, e.g., due to, for example, electrostatic, hydrophobic, ionic, pi-stacking, coordinative, van der Waals, covalent and/or hydrogen-bond interactions under physiological conditions.

As used herein, two nucleic acid sequences “complement” one another or are “complementary” to one another if they base pair one another at each position.

The term “modulation” or “modulate”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity, or process. In certain instances, such regulation may be contingent on the occurrence of a specific event, such as activation of a signal transduction pathway, and/or may be manifest only in particular cell types.

As used herein, “specific binding” refers to the ability of an aptamer to bind to a predetermined target. Typically, an aptamer specifically binds to its target with an affinity corresponding to a K_{D} of about 10⁻⁷ M or less, about 10⁻⁸ M or less, or about 10⁻⁹ M or less and binds to the target with a K_{D} that is significantly less (e.g., at least 2 fold less, at least 5 fold less, at least 10 fold less, at least 50 fold less, at least 100 fold less, at least 500 fold less, or at least 1000 fold less) than its affinity for binding to a non-specific and unrelated target (e.g., BSA, casein, or an unrelated cell, such as an HEK 293 cell or an E. coli cell).

The terms “oligonucleotide” and “nucleic acid molecule” refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

### Aptamers

In certain aspects, provided herein are aptamers that bind to T cells (e.g., CD8+ T cells) and/or induces T cell-mediated cytotoxicity. In some aspects, provided herein are pharmaceutical compositions comprising such aptamers, methods of using such aptamers to treat cancer and/or to kill cancer cells and methods of making such aptamers.

In certain aspects, provided herein are aptamers comprising a nucleic acid sequence that is at least 60% identical (e.g., at least 65% identical, at least 70% identical, at least 75% identical, at least 80% identical, at least 85% identical, at least 90% identical, at least 92% identical, at least 94% identical, at least 96% identical, at least 98% identical) to any one of SEQ ID NOs: 1-39, 59-77 or 80 (Tables 11 and 16-18). In certain embodiments, the aptamers comprise at least 20 (e.g., at least 25, at least 30, at least 35, at least 40, at least 41, at least 42, at least 43, at least 44, at least 45, at least 46, at least 47, at least 48, at least 49, at least 50) consecutive nucleotides of any one of SEQ ID NO: 1-27. In certain embodiments, the aptamers comprise at least 40 (e.g., at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, or at least 73) consecutive nucleotides of any one of SEQ ID NO: 28-39. In some embodiments, the aptamers comprise a nucleic acid sequence of any one of SEQ ID NOs: 1-39, 59-77 or 80 (e.g., any one of SEQ ID NOs: 3, 5, 6, 28, 59, 80, and 29). In some embodiments, the aptamers provided herein have a sequence consisting essentially of any one of SEQ ID NOs: 1-39, 59-77 or 80 (e.g., any one of SEQ ID NOs: 3, 5, 6, 28, 59, 80, and 29). In certain embodiments, the aptamers provided herein have a sequence consisting of any one of SEQ ID NO: 1-39, 59-77 or 80 (e.g., any one of SEQ ID NOs: 3, 5, 6, 28, 59, 80, and 29).

The terms “identical” or “percent identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like).

In certain embodiments, the aptamers are no more than 100 nucleotides in length (e.g., no more than 90 nucleotides in length, no more than 85 nucleotides in length, no more than 80 nucleotides in length, no more than 75 nucleotides in length, no more than 70 nucleotides in length, no more than 65 nucleotides in length, no more than 60 nucleotides in length, no more than 59 nucleotides in length, no more than 58 nucleotides in length, no more than 57 nucleotides in length, no more than 56 nucleotides in length, no more than 55 nucleotides in length, no more than 54 nucleotides in length, no more than 53 nucleotides in length, no more than 52 nucleotides in length, no more than 51 nucleotides in length, or no more than 50 nucleotides in length.

In some embodiments, the aptamers provided herein are able to bind to a T cell (e.g., a CD8+ T cell). In some embodiments, the aptamers provided herein bind to a T cell antigen selected from Notch 2 and other Notch family members, KCNK17, CD3, CD28, 4-1BB, CTLA-4, ICOS, CD40L, PD-1, OX40, LFA-1, CD27 PARP16, IGSF9, SLC15A3 and WRB. In some embodiments, the aptamers are able to induce T cell-mediated cytotoxicity. In some embodiments, the aptamers are able to induce cell death of a cancer cell (e.g., a human cancer cell) through T cell-mediated cytotoxicity. In some embodiments, the cancer cell is a patient-derived cancer cell. In some embodiments, the cancer cell is a solid tumor cell. In certain embodiments, the cancer cell is a colorectal carcinoma cell. In some embodiments, the cancer cell is a breast cancer cell. In some embodiments, the aptamers induce cell death of a cancer cell in vitro. In certain embodiments, the aptamers induce cell death of a cancer cell in vivo (e.g., in a human and/or an animal model).

In some embodiments, the aptamers provided herein comprise one or more chemical modifications. Exemplary modifications are provided in Table 1.

TABLE 1
Exemplary chemical modifications.

| Terminal | Sugar ring | Nitrogen base | Backbone |
|---|---|---|---|
| biotin | 2′-OH | BzdU | Phosphorothioate |
| | (RNA) | | |
| Inverted-dT | 2′-OMe | Naphtyl | Methylphosphorothioate |
| PEG (0.5-40 kDa) | 2′-F | Triptamino | Phosphorodithioate |
| Cholesterol | 2′-NH2 | Isobutyl | Triazole |
| Albumin | LNA | 5-Methyl Cytosine | Amide (PNA) |
| Chitin (0.5-40 kDa) | UNA | Alkyne | Alkyne |
| | | (dibenzocyclooctyne) | (dibenzocyclooctyne) |
| Chitosan (0.5-40 kDa) | 2′-F ANA | Azide | Azide |
| Cellulose (0.5-40 kDa) | L-DNA | Maleimide | Maleimide |
| Terminal amine | CeNA | | |
| (alkyne chain with | | | |
| amine) | | | |
| Alkyl | TNA | | |
| (dibenzocyclooctyne) | | | |
| Azide | I INA | | |
| Thiol | | | |
| Maleimide | | | |
| NHS | | | |

In certain embodiments, the aptamers comprise a terminal modification. In some embodiments, the aptamers are chemically modified with poly-ethylene glycol (PEG) (e.g., 0.5-40 kDa) (e.g., attached to the 5′ end of the aptamer). In some embodiments, the aptamers comprise a 5′ end cap (e.g., is an inverted thymidine, biotin, albumin, chitin, chitosan, cellulose, terminal amine, alkyne, azide, thiol, maleimide, NHS). In certain embodiments, the aptamers comprise a 3′ end cap (e.g., is an inverted thymidine, biotin, albumin, chitin, chitosan, cellulose, terminal amine, alkyne, azide, thiol, maleimide, NHS).

In certain embodiments, the aptamers provided herein comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) modified sugars. In some embodiments, the aptamers comprise one or more 2′ sugar substitutions (e.g. a 2′-fluoro, a 2′-amino, or a 2′-O-methyl substitution). In certain embodiments, the aptamers comprise locked nucleic acid (LNA), unlocked nucleic acid (UNA) and/or 2′deozy-2′fluoro-D-arabinonucleic acid (2′-F ANA) sugars in their backbone.

In certain embodiments, the aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50, 51, 52, 53, or 54) methylphosphonate internucleotide bonds and/or a phosphorothioate (PS) internucleotide bonds. In certain embodiments, the aptamers comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 3031, 32, 33, 34, 35, 36, 37, 38, 39, 40, 41, 42, 43, 44, 45, 46, 47, 48, 49, 50, 51, 52, 53, or 54) triazole internucleotide bonds. In certain embodiments, the aptamers are modified with a cholesterol or a dialkyl lipid (e.g., on their 5′ end).

In some embodiments, the aptamers comprise one or more modified bases (e.g., BzdU, Naphtyl, Triptamino, Isobutyl, 5-Methyl Cytosine, Alkyne (dibenzocyclooctyne, Azide, Maleimide).

In certain embodiments, the aptamers provided herein are DNA aptamers (e.g., D-DNA aptamers or enantiomer L-DNA aptamers). In some embodiments, the aptamers provided herein are RNA aptamers (e.g., D-RNA aptamers or enantiomer L-RNA aptamers). In some embodiments, the aptamers comprise a mixture of DNA and RNA.

Aptamers may be synthesized by methods which are well known to the skilled person. For example, aptamers may be chemically synthesized, e.g. on a solid support. Solid phase synthesis may use phosphoramidite chemistry. Briefly, the synthesis cycle starts with the removal of the acid-labile 5′-dimethoxytrityl protection group (DMT, “Trityl”) from the hydroxyl function of the terminal, support-bound nucleoside by UV-controlled treatment with an organic acid. The exposed highly-reactive hydroxyl group is then available to react in the coupling step with the next protected nucleoside phosphoramidite building block, forming a phosphite triester backbone. Next, the acid-labile phosphite triester backbone is oxidized to the stable pentavalent phosphate trimester. If a phosphorothioate modification is desired at a specific backbone position, the acid labile phosphite trimester backbone is sulfuridized at this step, instead of the oxidation process, to generate a P═S bond rather than a P═O. Successively, all the unreacted 5′-hydroxyl groups are acetylated (“capped”) in order to block these sites during the next coupling step, avoiding internal mismatch sequences. Following the capping step, the cycle starts again by removal of the DMT-protection group and successive coupling of the next base according to the desired sequence. Finally, the oligonucleotide is cleaved from the solid support and all protection groups are removed from the backbone and bases.

### Aptamer Conjugate

In certain aspects, provided herein are aptamer conjugates comprising an aptamer provided herein linked to a cancer cell-binding moiety. The cancer cell-binding moiety may be, e.g., an aptamer, a small molecule, a polypeptide, a nucleic acid, a protein, or an antibody. In some embodiments, the aptamer is covalently linked to the cancer cell-binding moiety. In some embodiments, the aptamer is non-covalently linked to the cancer cell-binding moiety. In some embodiments, the aptamer is directly linked to the cancer cell-binding moiety. In some embodiments, the aptamer is linked to the cancer cell-binding moiety via a linker.

In some embodiments, the cancer-cell binding moiety binds to an antigen expressed on a cancer cell. In some embodiments, the cancer-cell binding moiety binds to a cancer antigen selected from Prostate-specific antigen (PSA), Prostate Membrane Antigen (PSMA) Cancer antigen 15-3 (CA-15-3), Carcinoembryonic antigen (CEA), Cancer antigen 125 (CA-125), Alpha-fetoprotein (AFP), NY-ESO-1, MAGEA-A3, WT1, hTERT, Tyrosinase, gp100, MART-1, melanA, B catenin, CDC27, HSP70-2-m, HLA-A2-R170J, AFP, EBV-EBNA, HPV16-E7, MUC-1, HER-2/neu, Mammaglobin-A or MHC-TAA peptide complexes

In some embodiments, the cancer-cell binding moiety induces cell death (e.g., apoptosis) when contacted to a cancer cell (e.g., a human cancer cell). In some embodiments, the cancer cell is a patient-derived cancer cell. In some embodiments, the cancer cell is a solid tumor cell. In certain embodiments, the cancer cell is a colorectal carcinoma cell. In some embodiments, the cancer cell is a breast cancer cell. In some embodiments, the cancer-cell binding moiety induces cell death when contacted to a cancer cell in vitro. In certain embodiments, the cancer-cell binding moiety induces cell death when contacted to a cancer cell in vivo (e.g., in a human and/or an animal model).

### Pharmaceutical Compositions

In certain aspects, provided herein are pharmaceutical compositions comprising an aptamer (e.g., a therapeutically effective amount of an aptamer) provided herein. In certain aspects, provided herein are pharmaceutical compositions comprising an aptamer conjugate (e.g., a therapeutically effective amount of an aptamer conjugate) provided herein. In some embodiments, the pharmaceutical compositions provided herein further comprise a pharmaceutically acceptable carrier. In some embodiments, the pharmaceutical compositions provided herein are formulated for parenteral administration (e.g., subcutaneous administration). The administration may be an intratumoral injection or a peritumoral injection.

In certain embodiments, the pharmaceutical compositions are for use in treating cancer. In some embodiments, the cancer is a solid tumor. In certain embodiments, the cancer is a carcinoma (e.g., a colorectal carcinoma). In some embodiments, the cancer is a breast cancer.

“Pharmaceutically acceptable carrier” refers to a substance that aids the administration of an active agent to and absorption by a subject and can be included in the compositions described herein without causing a significant adverse toxicological effect on the patient. Non-limiting examples of pharmaceutically acceptable excipients include water, NaCl, normal saline solutions, Phosphate-buffered solution, MgCl₂, KCl, CaCl₂), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylase or starch, fatty acid esters, lipids, hydroxymethy cellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions described herein. One of skill in the art will recognize that other pharmaceutical excipients are useful.

### Therapeutic Methods

In some aspects, provided herein are methods of treating cancer comprising the administration of a pharmaceutical composition comprising one or more aptamers provided herein.

In some aspects, provided herein are methods of treating cancer comprising the administration of a pharmaceutical composition comprising one or more aptamer conjugates provided herein.

In some embodiments, the cancer is a solid tumor. In certain embodiments, the cancer is a colorectal carcinoma. In some embodiments, the cancer is a breast cancer. Thus, in certain aspects, provided herein is a method of delivering an aptamer, an aptamer conjugate, and/or a pharmaceutical composition described herein to a subject.

In certain embodiments, the pharmaceutical compositions, aptamers and aptamer conjugates described herein can be administered in conjunction with any other conventional anti-cancer treatment, such as, for example, radiation therapy and surgical resection of the tumor. These treatments may be applied as necessary and/or as indicated and may occur before, concurrent with or after administration of the pharmaceutical compositions, aptamers, aptamer conjugates, dosage forms, and kits described herein.

In certain embodiments, the method comprises the administration of multiple doses of the aptamer or aptamer conjugate. Separate administrations can include any number of two or more administrations (e.g., doses), including 2, 3, 4, 5, 6, 7, 8, 9, 10, 11, 12, 13, 14, 15, 16, 20, 21, 22, 23, 24, or 25 administrations. In some embodiments, at least 8, 9, 10, 11, 12, 13, 14, or 15 administrations are included. One skilled in the art can readily determine the number of administrations to perform, or the desirability of performing one or more additional administrations, according to methods known in the art for monitoring therapeutic methods and other monitoring methods provided herein. Accordingly, the methods provided herein include methods of providing to the subject one or more administrations of an aptamer, an aptamer conjugate and/or a pharmaceutical composition described herein, where the number of administrations can be determined by monitoring the subject, and, based on the results of the monitoring, determining whether or not to provide one or more additional administrations. Deciding on whether or not to provide one or more additional administrations can be based on a variety of monitoring results, including, but not limited to, cytotoxic activity of T cells, indication of tumor growth or inhibition of tumor growth, appearance of new metastases or inhibition of metastasis, the subject's anti-tumor antibody titer, the overall health of the subject and/or the weight of the subject.

The time period between administrations can be any of a variety of time periods. In some embodiments, the doses may be separated by at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days or 1, 2, 3, or 4 weeks. The time period between administrations can be a function of any of a variety of factors, including monitoring steps, as described in relation to the number of administrations, the time period for a subject to mount an immune response and/or the time period for a subject to clear the aptamers or aptamer conjugates from normal tissue. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the aptamers or aptamer conjugates from normal tissue; for example, the time period can be more than the time period for a subject to clear the aptamers or aptamer conjugates from normal tissue, such as more than about an hour, more than about a day, more than about two days, more than about three days, more than about five days, or more than about a week; in another example, the time period can be less than the time period for a subject to clear the aptamers or aptamer conjugates from normal tissue, such as less than about an hour, less than about a day, less than about two days, less than about three days, less than about five days, or less than about a week.

The administered dose of an aptamer or an aptamer conjugate described herein is the amount of the aptamer or the aptamer conjugate that is effective to achieve the desired therapeutic response for a particular patient, composition, and mode of administration, with the least toxicity to the patient or the maximal feasible dose. The effective dosage level can be identified using the methods described herein and depends upon a variety of pharmacokinetic factors including the activity of the particular compositions administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. In general, an effective dose of a cancer therapy is the amount of the therapeutic agent which is the lowest dose effective to produce a therapeutic effect. Such an effective dose generally depends upon the factors described above.

Examples of routes of administration include oral administration, rectal administration, topical administration, inhalation (nasal) or injection. Administration by injection includes intravenous (IV), intratumoral (IT), intralesional, peritumoral, intramuscular (IM), and subcutaneous (SC) administration. The compositions described herein can be administered in any form by any effective route, including but not limited to oral, parenteral, enteral, intravenous, intratumoral, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In some embodiments, the aptamers or aptamer conjugates described herein are administered orally, rectally, topically, intravesically, by injection into or adjacent to a draining lymph node, intravenously, by inhalation or aerosol, or subcutaneously. In some embodiments, the administration is parenteral administration (e.g., subcutaneous administration). The administration may be an intratumoral injection or a peritumoral injection.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, tumor dimensions, general health and specific biomarkers, the particular microorganism to be administered, duration and route of administration, the kind and stage of the disease, for example, tumor size, and other compounds such as drugs being administered concurrently.

The methods of treatment described herein may be suitable for the treatment of a primary tumor, a secondary tumor or metastasis, as well as for recurring tumors or cancers. The dose of the pharmaceutical compositions described herein may be appropriately set or adjusted in accordance with the dosage form, the route of administration, the degree or stage of a target disease, and the like.

In some embodiments, the dose administered to a subject is sufficient to prevent cancer, delay its onset, or slow or stop its progression or prevent a relapse of a cancer, reduce tumor burden, or contribute to the disease-free survival, time to progression or overall survival of the subject. One skilled in the art will recognize that dosage will depend upon a variety of factors including the strength of the particular compound employed, as well as the age, species, condition, and body weight of the subject. The size of the dose will also be determined by the route, timing, and frequency of administration as well as the existence, nature, and extent of any adverse side-effects that might accompany the administration of a particular compound and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting, e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the aptamers or aptamer conjugates provided herein may vary depending on the specific aptamer or aptamer conjugate, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and most preferably causing complete regression of the cancer.

Examples of cancers that may treated by methods described herein include, but are not limited to, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, rhabdosarcoma, serocystic sarcoma, synovial sarcoma, telangiectaltic sarcoma, Hodgkin's Disease, Non-Hodgkin's Lymphoma, multiple myeloma, neuroblastoma, bladder cancer, breast cancer, ovarian cancer, lung cancer, colorectal cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, superficial spreading melanoma, plasmacytoma, colorectal cancer, rectal cancer.

In some embodiments, the methods and compositions provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and compositions described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

Particular categories of tumors that can be treated using methods and compositions described herein include lymphoproliferative disorders, breast cancer, ovarian cancer, prostate cancer, cervical cancer, endometrial cancer, bone cancer, liver cancer, stomach cancer, colon cancer, colorectal cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

Cancers treated in certain embodiments also include precancerous lesions, e.g., actinic keratosis (solar keratosis), moles (dysplastic nevi), acitinic chelitis (farmer's lip), cutaneous horns, Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

In certain embodiments, the cancer is a solid tumor (e.g., breast cancer, head and neck squamous cell carcinoma, adenoid cystic carcinoma, bladder cancer, pancreatic cancer, hepatocellular carcinoma, melanoma, merkel cell carcinoma, or a colorectal carcinoma). In some embodiment, the solid tumor is accessible for intratumoral administration. In certain embodiment, the cancer is a sarcoma (e.g., soft tissue sarcoma). In certain embodiments, the cancer is a hematologic cancer (e.g., a lymphoma).

### EXAMPLES

### Example 1—Materials and Methods for Examples 2-3

A. Materials

a. Random Library

Random library 2.6 was purchased from IDT. The library contains a vast repertoire of approximately 10¹⁵ different 50 nt-long random sequences flanked by two unique sequences at the 3′ and 5′ acting as primers for PCR amplification during the SELEX procedure. The lyophilized library (“Lib 2.6”) was reconstituted in ultra-pure water (UPW) to a final concentration of 1 mM. The random library sequence was: 5′TATCCGTCTGCTCTCGCTATNN NNNNNNNNNNNNNNNNNNACGCACCTAATGTCCTACTG-3′ (SEQ ID NO: 43), where N represents a random oligonucleotide selected from a mixture of equally represented T, A, C and G nucleotides.

b. Pre-SELEX Preparation

Library 2.6 (Lib 2.6) underwent QC validation using HPLC gel filtration column.

c. Library Primers and Caps

A set of 20 nt primers and caps were purchased from IDT. Caps were used to hybridize to the Library's primer sites during incubation with cells in order to refrain from the possibility of primer sequences interacting with the random 50 nt sequence site. A mixture of 3′ and 5′ caps in each SELEX round was used in a 3:1 caps-to-library ratio.

The forward primer was purchased from IDT labelled with Cy-5 at the 5′ site for sequence amplification that was detected in a fluorescence assay. The lyophilized primers were reconstituted in ultra-pure water (UPW) to a final concentration of 100 μM.

TABLE 2
Random library, primers and caps sequences

| | SEQ | |
|---|---|---|
| | ID | |
| | NO: | Sequence 5′ to 3′ |
| Random | 43 | TATCCGTCTGCTCTCGCTATNN |
| Library | | NNNNNNNNNNNNNNNNNNNNNN |
| | | NNNNNNNNNNNNNNNNNNNNNN |
| | | NNNACGCACCTAATGTCCTACT |
| | | G |
| Forward | 44 | TATCCGTCTGCTCTCGCTAT |
| Primer | | |
| Reverse/3′ | 45 | CAGTAGGACATTAGGTGCGT |
| cap | | |
| 5′ cap | 46 | ATAGCGAGAGCAGACGGATA |
| Forward | 47 | /5Cy5/TATCCGTCTGCT |
| labelled Cy-5 | | CTCGCTAT |

d. Aptamer Folding Buffer

Phosphate-buffered saline (minus Magnesium and Calcium) was supplemented with 1 mM Magnesium Chloride (MgCl₂). The folding buffer was sterilized with PVDF membrane filter unit 0.22 μm and kept at 4° C.

e. Fresh PBMC

Blood samples were obtained from Tel Hashomer medical center blood bank and PBMC were isolated using Ficoll (Lymphoprep, Axis-Shield) density gradient centrifugation following the manufacturer's protocol.

f. Human CD8 T Cell Isolation

Isolation of human CD8 cells was performed via CD8+ T cells isolation Kit (Miltenyi Biotec, 130-096-495) following the manufacturer's protocol.

g. Aptamers List

Each aptamer was diluted to the desired concentration with the folding buffer. The aptamers were heated for 5 minutes at 95° C., followed by a rapid cooling for 10 minutes on ice, and room temperature (RT) incubation for 10 minutes. Folded aptamer was then added to the medium-suspended cells.

The following aptamers were used:

TABLE 3
Sequences related to CTL3
identification as T cell engager

| | SEQ | |
|---|---|---|
| | ID | |
| Aptamer name | NO: | Sequence 5′ to 3′ |
| Poly T 5′-Cy5- | 48 | /5Cy5/TTTTTTTTTTTTTTT |
| labelled | | TTTTTTTTTTTTTTTTTTTTT |
| | | TTTTTTTTTTTTTT |
| RND aptamer 5′- | 49 | /5Cy5/CCGCGTCCGGACACC |
| Cy5-labelled: | | TAATTTGGTTCAAGAGCCGCC |
| | | CGTAATTTCAGGTTCTCC |
| CTL3 5′-Cy5- | 50 | /5Cy5/GCATACCTTTCGTAT |
| labelled | | GCCTTTTTGACCCGTATTTTT |
| | | GCCCTACCCTTCGG |
| Scrambled-CTL3-A | 51 | /5Cy5/GTTCTTATAATCGCC |
| 5′-Cy5-labelled: | | TCTGCGCTATGTTCTTGCTCG |
| | | CCTTCCATATCGCT |
| Scrambled-CTL3-A | 52 | GTTCTTATAATCGCCTCTGCG |
| | | CTATGTTCTTGCTCGCCTTCC |
| | | ATATCGCT |
| Scrambled-CTL3-B | 53 | TCTTTCGTTAGCGCTTCTCTC |
| | | TTGCGATTCCGACCGCATATT |
| | | CACGTCTT |

Lyophilized aptamers were kept in dark at RT until reconstituted in PBS-supplemented with 1 mM MgCl₂ to a concentration of 100 μM and stored at −20° C. in the dark.

B. Experimental Methods

a. Binding SELEX Protocol

The binding SELEX was conducted for 7 sequential rounds using CD8⁺ cells isolated from three healthy donors including two negative selections rounds (after rounds 3 and 4). The binding SELEX was performed as follows:

(1) Isolation and Preparation of CD8 Cells for Individual SELEX Round

Prior to each round, CD8 cells were isolated, and recovered for 1 hour in a warm RPMI1640 (ATCC) at 37° C. Subsequently, cells were counted and seeded in a 1.5 mL Eppendorf tube at the following concentration:

TABLE 4
Amount of CD8 cells and negative selection cells in each binding SELEX round

| | Round | Round | Round | Round 3 | Round | Round 4 | Round | Round | Round |
|---|---|---|---|---|---|---|---|---|---|
| | 1 | 2 | 3 | negative | 4 | negative | 5 | 6 | 7 |
| Amount of | 10 × 10⁶ | 7 × 10⁶ | 4 × 10⁶ | | 3 × 10⁶ | | 2 × 10⁶ | 1 × 10⁶ | 1 × 10⁶ |
| CD8 cells | | | | | | | | | |
| Amount of | | | | 10 × 10⁶ | | 3 × 10⁶ | | | |
| CD8 | | | | | | | | | |
| negative | | | | | | | | | |
| cells | | | | | | | | | |

(2) Initial Library and Enriched Round Library Preparation and Folding Protocol

The library is initially reconstituted to 1 mM. Working concentration in the first round was 14.3 μM, while in rounds 2-7, a concentration of 0.25-0.5 μM of enriched library was used. For each round the following components were used:

TABLE 5
Calculating library concentrations

| Component | Concentration | Calculation |
|---|---|---|
| Enriched library | 0.25-0.5 μM | $\begin{matrix}{C}_{μ M} = \frac{{C}_{\frac{ng}{μ l}} \times 1 \text{,} 000}{330 gr / mole \times [ 90 nt ( lib length ) ]} \\ {C}_{μ M} \times {V}_{Elution} = [ 0.2 up to 0.5 ] μM \times {V}_{pool}\end{matrix}$ |
| Mix caps 5’ + 3’ | 50 uM | $\frac{{V}_{Elution} \times {C}_{Elution}}{5 {0}_{μ M}} \times 3 = {V}_{mix caps}$ |
| Folding buffer | X10 | $\frac{{V}_{Elution} + {V}_{mix caps}}{9} = {V}_{FBX 10}$ |

The libraries underwent DNA folding per the following protocol: were heated for 5 minutes at 95° C., followed by a rapid cooling for 10 minutes on ice, and room temperature (RT) incubation for 10 minutes. After folding, the following components were added in order to avoid non-specific nucleotide absorption and adjusted to a final volume as in Table 6:

TABLE 6
Calculating supplements

| | | | Final |
|---|---|---|---|
| Component | Concentration | Volume | concentration |
| tRNA | 10 mg/ml | 3.5 μl | 0.1 mg/ml |
| NaN₃ | 10% (in PBS) | 3.5 μl | 0.1% |
| Medium + | N.A. | Adjust volume to | N.A. |
| 10% serum | | 350 μl | |

(3) SELEX Round Duration and Washing Conditions

Once the enriched library round was folded, it was added to the isolated CD8 cells or to the negative cell population for a period of time as follows:

TABLE 7
Incubation time for each binding SELEX round

| | Round | Round | Round | Round 3 | Round | Round 4 | Round | Round | Round |
|---|---|---|---|---|---|---|---|---|---|
| | 1 | 2 | 3 | negative | 4 | negative | 5 | 6 | 7 |
| Positive | 1 h | 50 min | 40 min | | 30 min | | 20 min | 15 min | 15 min |
| SELEX | | | | | | | | | |
| Negative | | | | 1 h | | 1 h | | | |
| SELEX | | | | | | | | | |

After incubation, the cells were washed three times and centrifuged at 300 g for 5 min and the supernatant, “unbound to positive” fraction, was removed kept at −20° C. until NGS preparation. Cells were re-suspended with binding buffer and washed again. After the third wash, the cells were re-suspended in UPW, or binding buffer if a negative SELEX round was followed, and cells were lysed by heating for 95° C. for 10 min and centrifuged at full speed for 5 min at RT. The supernatant, “bound to positive” fraction, was removed, and used as a template for PCR reaction. If a negative SELEX round was followed, then the bound fraction was applied on CD8 negative cells for 1 hour at the same conditions described above and the collected fractions were called “unbound to negative” and “bound to negative”, respectively. After a negative SELEX round, the faction that was used for PCR amplification was the “unbound to negative” one.

(4) PCR Amplification Protocol

The “bound to positive” or the “unbound to negative” fraction was used as a template for asymmetrical PCR amplification. The PCR reaction was modulated for each round. The PCR components and the amplification protocol are shown in table 8 and table 9, respectively.

TABLE 8
PCR components

| Reagent | Stock | Volume |
|---|---|---|
| UPW | | Adjust to reaction final |
| | | volume |
| Buffer | x5 | Adjust to reaction final |
| | | volume |
| dNTPs mix | 10 mM | |
| Forward primer | 10 μM | |
| Reverse primer | 10 μM | |
| Template | | 10%-20% |
| DNA polymerase | | 1% |
| enzyme | | |

TABLE 9
PCR amplification protocol for enriched library

| Number of | | |
|---|---|---|
| cycles | Temperature | Duration |
| 1 | 95° C. | 3 min |
| 30-36 | 95° C. | 30 sec |
| | Primer Tm-5° C. | 30 sec |
| | 68° C. | 30 sec |
| 1 | 68° C. | 4 min |

(5) PCR ssDNA Purification

The PCR products were purified using HPLC or by PCR ssDNA gel extraction kit (QIAEX II) followed by the manufacturer's protocol. After purification, the DNA concentration was measured using NanoDrop, and the DNA was diluted for a new SELEX round.

b. SELEX Libraries Binding Assay Protocol

Isolated CD8 cells or CD8-negative cell fraction (negative control) were counted, and 1×10⁶ cells were divided each into 1.5 mL eppendorf tube. Cells were centrifuge and washed once with binding buffer. The cells were re-suspended in 225 μL binding buffer supplemented with 0.01% Azide and 0.1% tRNA, and 25 μL folded Cy5-labelled aptamers were added to each treatment, followed by 1 hour incubations at 37° C. in the dark. Cells were washed 4 times with binding buffer supplemented with 0.01% Azide and 0.1% tRNA, and fluorescence intensity was measured after each wash using flow cytometry (CytoFlex).

c. Individual Aptamers Binding Assay

Isolated CD8 cells or Pan T cells, PBMCs or cell-line were counted, and 1×10⁶ cells were divided into each 1.5 mL eppendorf tube. Cells were centrifuge and washed once with binding buffer. The cells were re-suspended in 225 μL RPMI1640 supplemented with 10% human serum, and the folded Cy5-labelled aptamers were added to each treatment, followed by 1 hour incubations on ice in the dark. Cells were washed 4 times with cold medium and fluorescence intensity was measured using flow cytometry (CytoFlex).

d. Thermofluorimetric Analysis (TFA)

TFA was used to determine the binding of CTL3 with its putative target Notch2.100 nM CTL3, 1 uM SYBR green I (sigma), Fc-Notch2 human (R&D Systems) or Fc-CD160 (abeam) at 20, 40, 80, 160, and 320 nM were mixed together and SYBR green and fluorescence was measured from Temp=25° C. to Temp=95° C. at 1 degree/min using RT-PCR, in triplicates. The subsequent experiment was done with 50 nM of either CTL3 (SEQ ID NO: 3), scrambled-CTL3-A (SEQ ID NO: 52), or scrambled-CTL3-B (SEQ ID NO: 53); 1 μM SYBR green I (sigma); Fc-Notch2 human (R&D Systems), Fc-Notch2 mouse (R&D Systems) or Fc-Notch2 rat at 25, 50, 100 and 200 nM similar to the former experiment.

### Example 2—Identification of T Cell Engager Candidates Via Binding SELEX

T cells have been established as core effectors for cancer immunotherapy, especially owing to their abundance, killing efficacy, and capacity to proliferate. T-cell engagers are bispecific molecules directed against a constant-component of the T-cell/CD3 complex on one end and a tumor-expressed ligand or antigen on the other end. This structure allows a bispecific T cell engager to physically link a T cell to a tumor cell, ultimately stimulating T cell activation and subsequent tumor killing (Huehls et al. (2015) Immunol. Cell Biol. 93:290-296; Ellerman D. (2019) Methods 154:102-117).

Selection of the Cytotoxic T Lymphocyte engaging aptamers was described herein. The cytotoxic T-lymphocyte arm was generated via Binding Cell-Selex using samples from multiple blood donors. The final lead was characterized for its binding to the target CD8+ T-Cells, its putative protein target identified via membrane protein array assay and was validated via thermofluorimetric analysis.

This disclosure describes the identification and characterization of the cytotoxic T lymphocyte (CTL) engaging aptamers from a random library of 10¹⁵ potential aptamers using the Cell-SELEX methodology in a novel application. This arm was designed to be constant across different patients.

In SELEX protocol, CTLs isolated from multiple healthy donors were used, sequentially in iterative selection rounds, to increase the likelihood of identifying aptamers that target widespread ligands, as oppose to individually-unique isoforms/mutants. To increase the specificity of the aptamer pool towards CTLs, negative selection was added in the form of CD8-negative PBMCs. In the final round of Cell-Selex, washing stringency of bound aptamer population was increased both in duration and in number of washes, in order to increase the affinity of potential aptamers in the final pool. After sequencing via next generation sequencing (NGS) and statistical analysis of enriched libraries throughout the selection process, putative binders were screened individually for their ability to bind primary CTLs. Top leads were tested for their capacity to promote target cancer cell cytotoxicity in the assembled structure of the bispecific aptamer, carrying a cancer-targeting aptameric arm. Concomitantly, Membrane Protein Array (MPA) platform (Tucker et al. (2018) Proc. Natl. Acad Sci. U.S.A. 115:E4990-E4999) was used to deconvolute the putative targets of top leads, and the target of one leading aptamer “CTL3” was further validated, using thermofluorimteric analysis (Hu, Kim, & Easley (2015) Anal. Methods 7:7358-7362). The target of CTL3 was shown to be Notch-2, a membrane signaling receptor implicated in T-Cell-Mediated anti-tumor immunity and T-cell-based immunotherapy (Janghorban et al. (2018) Frontiers in Immunology 9:1649; Duval et al. (2015) Oncotarget 6:21787-21788; Ferrandino et al. (2018) Frontiers in Immunology 9:2165; Kelliher and Roderick (2018) Frontiers in immunology 9:1718; Weerkamp et al. (2006) Leukemia 20:1967-1977).

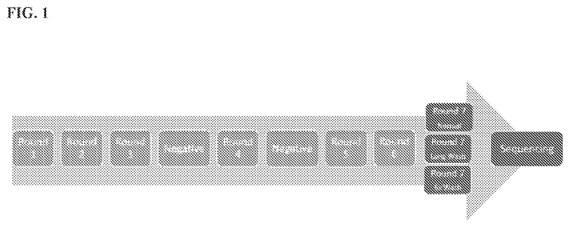

Binding Cell-SELEX was conducted using three healthy PBMCs donors for a total of seven rounds, as shown in FIG. 1. The use of multi PBMCs donors was carried out to ensure robustness of the aptamer-binding ability across different potential patients and not target a unique epitope expressed only in PBMCs of a single donor. Rounds 3 and 4 were followed by a negative selection round using CD8-negative PBMCs from donor 1 and 2.

a. SELEX Rounds Comparative Assay

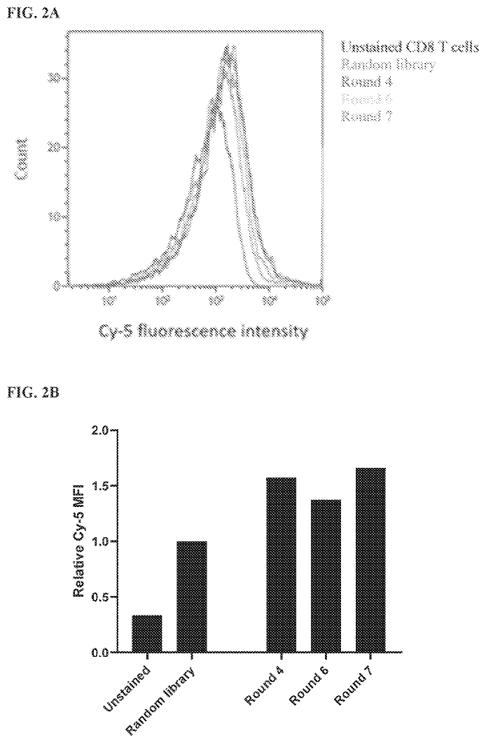

Libraries eluted from Rounds 4, 6 and 7 were tested for their binding affinity with isolated CD8 cells. Each round was amplified using 5′ primer labelled with Cy-5 followed by incubation with CD8 isolated cells for 1 hr. As shown in FIGS. 2A and 2B, the affinity of libraries from rounds 4, 6 and 7 was much higher than the random initial library used in the binding SELEX.

b. NGS Results

The final round of Binding Cell-SELEX was repeated two more times with increased wash stringencies, once doubling the number of washing of unbound sequences (“6× Wash”, relative to the baseline 3× Wash), and a second time with increased incubation time after the final wash to allow aptamers with high K_{off} to be released into the medium and washed out (“long wash”) (see Table 10).

Enriched libraries for the 2^{nd}, 5^{th}, 6^{th} and three conditions of the 7^{th} round were sequenced (“bound”), as well as the supernatant of each round (“unbound”), via high-throughput sequencing using NGS Illumina NextSeqg500.

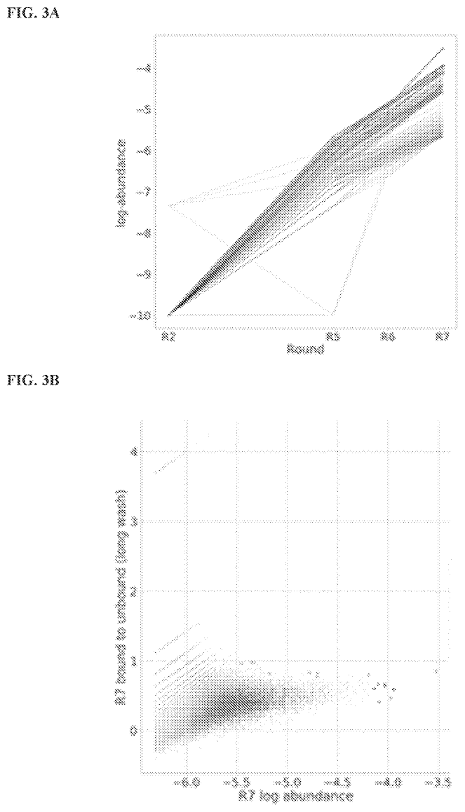

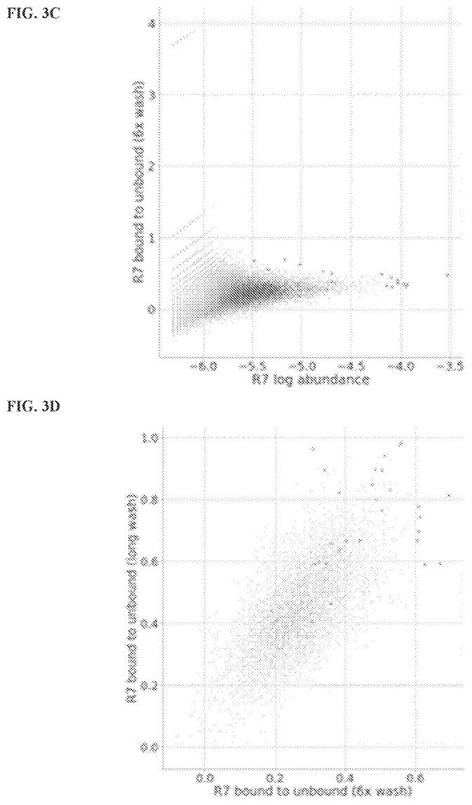

FIG. 3A shows the relative abundance of the most abundant sequences—the 10 most abundant in color and the rest in black (a total of 100 sequences). The results in FIG. 3A show increased abundance of top aptamers in the final enriched library, consistent with the increased binding results in FIGS. 2A and 2B.

Other than relative abundance, two additional measurements were calculated for each sequence in the final round 7 enriched library: the fraction of the sequence found in the cell bound population relative to the unbound population (supernatant) for the increased number of washes (6× Wash). The fraction of the sequence found in the cell bound population relative to the unbound population (supernatant) for the increased duration of the final wash (Long Wash).

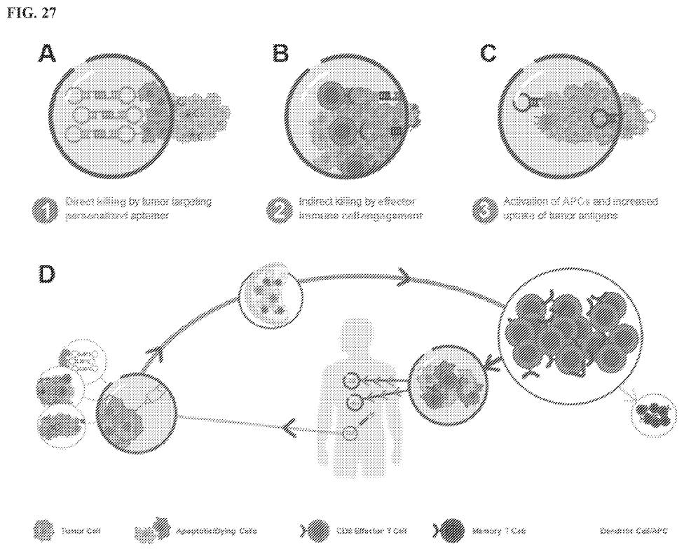

The three measurements for each sequence in the final enriched library were plotted against each other (FIGS. 3B-3D) and 27 sequences were picked to be synthesized and tested individually for their binding to CTLs (Table 11).

TABLE 10
Final Round permutations: wash stringency

| | Normal | 6 washes | Long wash |
|---|---|---|---|
| SELEX round | 15 min binding | 15 min binding | 15 min binding |
| duration | | | |
| Washes | 90 sec washX3 | 90 sec washX6 | 1. 90 sec washX2. |
| | | | 2. 30 min wash at 37° C. |
| Dissociation | 95° C. for 10 min | 95° C. for 10 min | 95° C. for 10 min |

TABLE 11
Tested CD81 binding aptamers

| | SEQ | |
|---|---|---|
| Aptamer | ID | |
| name | NO: | Sequence 5′ to 3′ |
| CTL1 | 1 | TACGCGCAATTCGCCTTGTCGGTGA |
| | | TCTTCCTTTGAACTTGGGCAGTCTG |
| CTL2 | 2 | TGGCCTGGCCGTGTCGTCTGCTTTA |
| | | TAGTCGGTGATCCCTTGTGTTAATT |
| CTL3 | 3 | GCATACCTTTCGTATGCCTTTTTGA |
| | | CCCGTATTTTTGCCCTACCCTTCGG |
| CTL4 | 4 | TTTTTCGCTATCCAACCCTTCTTTC |
| | | CAGCCTGCCAATCAGTCGGTGATCA |
| CTL5 | 5 | AGGGCAGTCCTGTATCTTAACATTC |
| | | TCCTACATCCGTAAGTCGGTGATCC |
| CTL6 | 6 | GGGCTAAGAGTCTCTATTGTCGGCA |
| | | GTCGTCTAATATTTCCCGTCCAATT |
| CTL7 | 7 | ACTTCCGGTGATTTGATTTCACTTC |
| | | CTGGGCAGTCAATGTGATTCTCTAC |
| CTL8 | 8 | GGTCAGTCGCCTTTGTCGGTGATGT |
| | | ACTCGCGCAGTCGGGTTCCCCTTAA |
| CTL9 | 9 | GGGTCTGTTGGTCCTAGGGCAGTCG |
| | | TACTTCTAATTCTTGTCCCGATGAT |
| CTL10 | 10 | CTTGTCGGTGATCTATAGTCGGTGA |
| | | TATATTTTGTCCTATGGTAGTCGAT |
| CTL11 | 11 | GGGCTCATGGGCAGTCTTTTTACTA |
| | | CCTCCTATTTACGTATCCCGCTCCT |
| CTL12 | 12 | CACCCGCGCATTTCCCCCCAGTCGG |
| | | TGATTCTTATATGTACCTGTTCCTC |
| CTL13 | 13 | GGGCACGTCCATTCGCGTTTTTGTT |
| | | CCGTTTCTCCCTTTTTGGATTTTGC |
| CTL14 | 14 | CAGTCGGTGTCACTCCAGCGGTCGG |
| | | TTCACTCCACATTCTCCCATCTGTC |
| CTL15 | 15 | GGCAGTCACCATTCTCTTTGGGCAG |
| | | ATTGTCTCTCATCCATATGTCTCCT |
| CTL16 | 16 | CTACCTCCTTAGTCGGTGATTCGAT |
| | | CTATGGGCCTAACTGCCTTCTCTGT |
| CTL17 | 17 | GGGATGCGGGGCCCCGTTCTTTTTG |
| | | TCTCTCATTTTGTCACTTTTTTTGT |
| CTL18 | 18 | GGTCAGTCCCTTCGGCATGTCGGGA |
| | | TTCCCTCTTTTCGCCTCGTTTCTTT |
| CTL19 | 19 | GGCTGTCGAACTTTCTCCCTCCCAC |
| | | CGCAGTCGGCCCCTCATCAGTCGTA |
| CTL20 | 20 | ACTTCCGGTGATTTGATTTCACTTC |
| | | CTGGGCAGTCAATGTGATTCTCTAC |
| CTL21 | 21 | ACGTCTGTCGGTGACCTGTAATAGT |
| | | TTATGTCGGTGATACAGCTTTCCCT |
| CTL22 | 22 | CTGTCGGTGATCATATAACGCAGTC |
| | | GGTGTAGTTTAATCCCACTCCCCTA |
| CTL23 | 23 | GGCCAGTGTCCCAGTCGTGATTGTA |
| | | ATATTAGATTCTTTGTGGCAGTCGT |
| CTL24 | 24 | ACTCGTCGGTGATTTTAGACCTTTC |
| | | TCGGTGATCAACACGTCATGCTATT |
| CTL25 | 25 | GCCTCGATATCCTCAGGAGTCGGTG |
| | | TTTCATTCAATCGTCGGTGATAAAT |
| CTL26 | 26 | GGTCAGTCCGTATACCGCCAATCCG |
| | | AACCGCAGTCGGTGTCCGCTTTTAC |
| CTL27 | 27 | TCGGGTTAGATGTCGGTCCCACTAT |
| | | ATGTCGGTGATCTAATATTGAACTT |

c. Individual Aptamers Validation

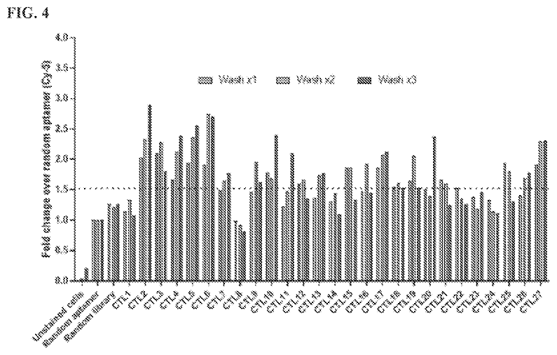

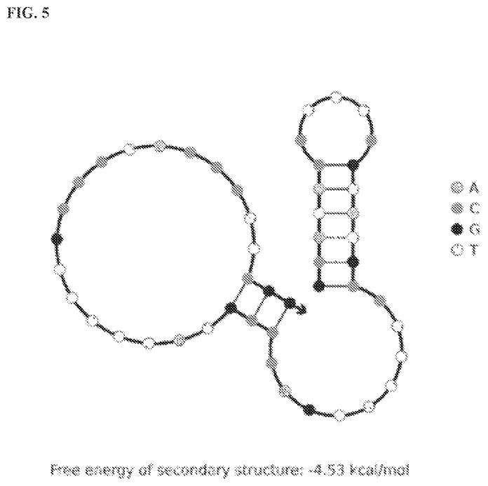

Aptamers selected from the statistical analysis were synthesized with a 5′ Cy5 fluorescence label and screened for their binding to isolated CD8 cells. A positive binding threshold was determined as above 1.5 folds over random aptamer sequence (FIGS. 4A and 4B).

### Example 3—T Cell Engager Characterization (of Example 2)

a. CTL3 Sequence and Structure

CTL3 sequence:
(SEQ ID NO: 3)
5′-GCATACCTTTCGTATGCCTTTTTGACC
CGTATTTTTGCCCTACCCTTCGG-3′

The predicted structure of CTL3 by Nupack software is shown in FIG. 5.

b. CTL3 Binding Assay Via Flow Cytometry

1. CTL3-Binding to Human PBMCs

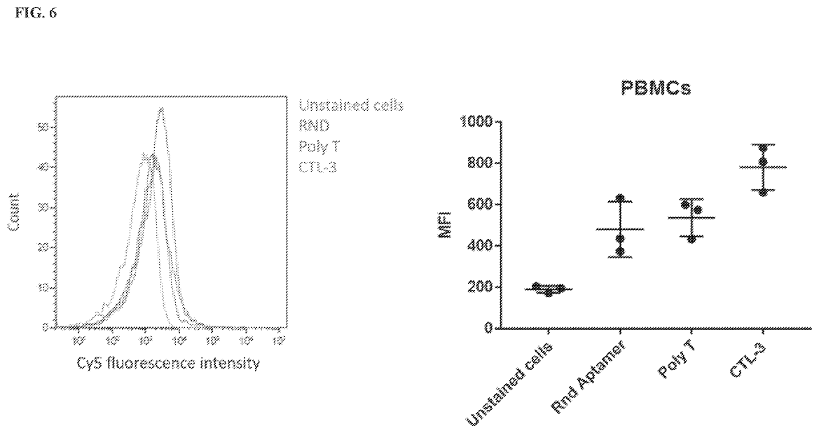

To visually demonstrate the binding of the selected aptamer to its target cell type and to better understand its specificity, human frozen PBMCs from several different donors were thawed and stained with CTL3 Cy5-labelled aptamer as well as Cy5-labelled negative controls, Poly-T and random (RND) aptamer sequence. CTL3 aptamer exhibited higher binding to total PBMCs compared with random aptamer control and Poly T aptamers (FIG. 6).

To better understand the specificity of CTL3 aptamer, CD8-staining was used together with SSC/FSC to differentiate between PBMC subpopulations.

Human PBMCs from three different healthy donors were tested for binding with Cy5-labelled aptamers (250 nM) followed by CD8 antibody staining.

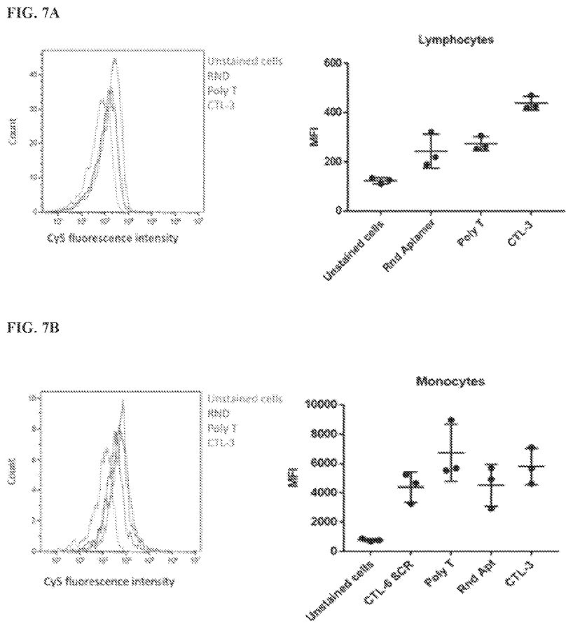

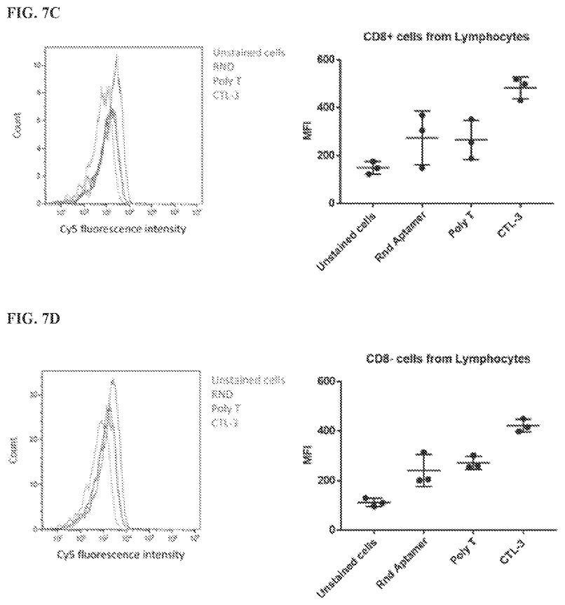

Binding of CTL3 to lymphocyte population was greater compared to RND control and Poly T aptamers, while no significant binding differences between CTL3, RND control and poly T aptamers to monocytes cells were observed (FIGS. 7A and 7B). Within the lymphocytic population however, CTL3 was found to bind both CD8-positive and CD8-negative lymphocytes (FIGS. 7C and 7D).

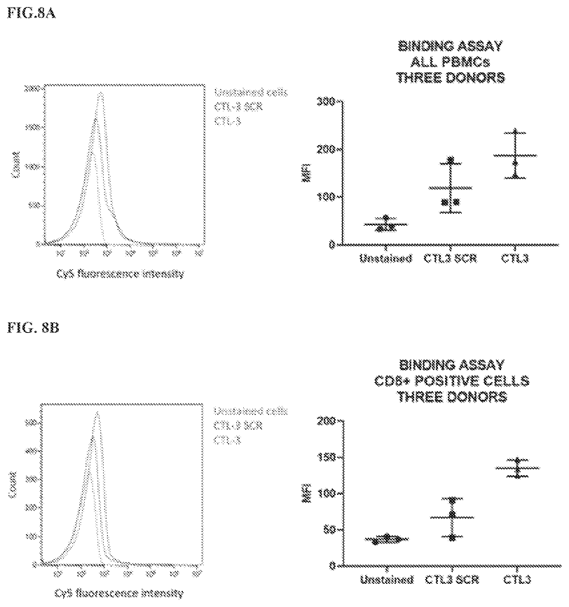

A scramble sequence (SCR) containing the same nucleotides ratio as CTL3 was designed. CTL3 demonstrated binding even in comparison with this stringent control (FIGS. 8A and 8B).

2. Binding Assay with Isolated CD8 Cells

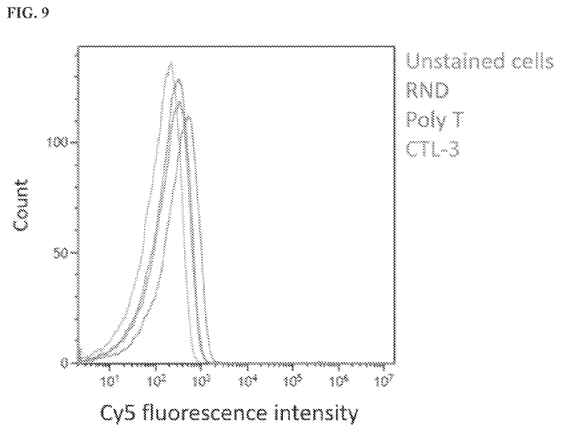

In order to rule out reduced signal due to a mixed PBMC population, CD8 T cells were isolated prior to the assay and CTL3 binding was measured directly on this subpopulation. FIG. 9 displayed representative results from a single experiment. The results nevertheless, were consistent with the PBMCs binding results.

3. CTL3 Binding to Expanded and Stimulated T Cells

CTL3 aptamer was subjected to target de-orphaning described herein, and Notch2 was identified and validated as the aptamer's target. Notch2 surface expression is dynamically regulated during T cell development and activation (Duval et al. (2015) Oncotarget 6:21787-21788; Ferrandino et al. (2018) Frontiers in Immunology 9:2165; Kelliher and Roderick (2018) Frontiers in immunology 9:1718; Weerkamp et al. (2006) Leukemia 20:1967-1977).

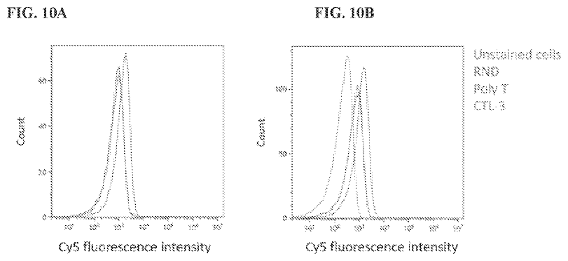

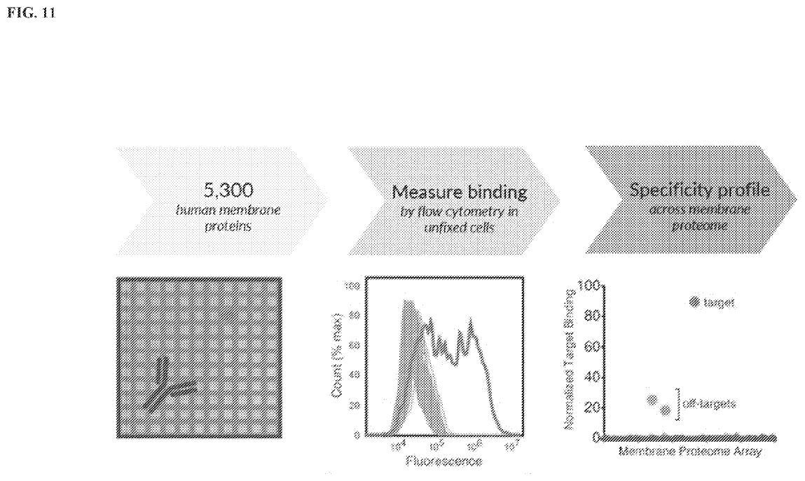

To measure the dependency of CTL3 binding on the active state of the target cells, an exploratory experiment was performed in which T cells were isolated from one donor's PBMCs, via pan-T isolation kit, and activated via a combination of anti-CD3 (1 μg/μL) & anti-CD28 (1 μg/μL) antibodies for 48 hr followed by IL-2 (300 Unit) for 9 days. Binding was measured 11 days after the initial activation. Under these conditions, no significant increase in CTL3 binding ability was observed compared to binding with all hPBMCs or isolated CD8 T cells (FIGS. 10A and 10B)

c. Target Deconvolution of CTL3 by Membrane Proteome Array

The Membrane Proteome Array (MPA) is a platform developed by Integral Molecular Inc (Philadelphia, Pa., US) for profiling the specificity of antibodies and other ligands that target human membrane proteins. The MPA can be used to determine target specificity and deconvolute orphan ligand targets (Tucker et al. (2018) Proc. Natl. Acad. Sci. U.S.A. 115:E4990-E4999).

The platform uses flow cytometry to directly detect ligand binding to membrane proteins expressed in unfixed cells (see FIG. 11). Consequently, all target proteins have native conformations and appropriate post-translational modifications.

CTL3 aptamer was tested for reactivity against a library of over 5,300 human membrane proteins, including 94% of all single-pass, multi-pass and GPI-anchored proteins. Identified targets were validated in secondary screens to confirm reactivity.

A high-throughput cell-based platform is used to identify the membrane protein targets of ligands. Membrane proteins are expressed in human cells within 384-well microplates, and ligand binding is detected by flow cytometry, allowing sensitive detection of both specific and off-target binding.

Each well on the matrix plate contains 48 different overexpressed protein constituents. Each protein is represented in a unique combination of two different wells of the matrix plate, as it is contained within a “row” pool and a “column” pool. Test CS aptamer was added to MPA matrix plates at predetermined concentrations, washed in 1×PBS, and detected by flow cytometry.

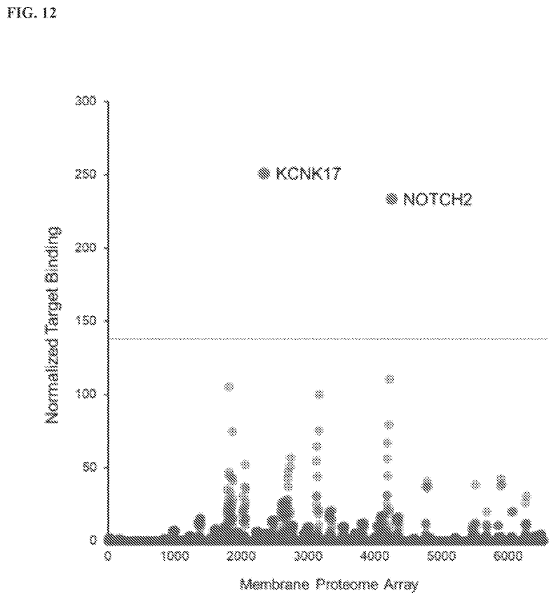

CTL3 aptamer target hits were then identified by detecting binding to overlapping pooled matrix wells emanating from the same transfection plate, thereby allowing specific deconvolution. The screening yielded two potential hits: KCNK17 and Notch2 (FIG. 12).

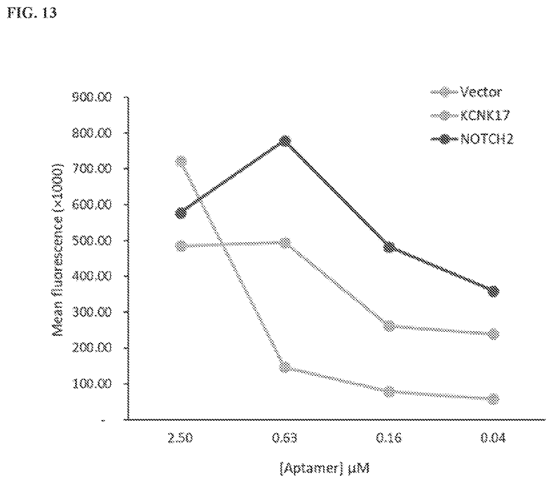

To validate protein targets identified using the MPA, HEK 293T cells were transfected with plasmids encoding the respective targets, or vector alone (pUC; negative control) in 384-well format. After incubation for 36 hours, four 4-fold dilutions of CTL3 were added to transfected cells followed by detection of aptamer binding using a high-throughput immunofluorescence flow cytometry assay. Average mean fluorescence intensity (MFI) values were determined for each aptamer dilution (FIG. 13). Notch2 and KCNK17 (a potassium channel subfamily K member 17) have been validated to generate a concentration-dependent binding curve substantially higher than the negative control vector's.

d. Binding of CTL3 to Recombinant Notch2 by Thermofluorimetric Analysis

While no T cell related literature was found for KCNK17, the Notch pathway regulates CD8 T cells in multiple ways. CD8-specific deletion of Notch2, but not Notch1 for example, led to increased tumor size and decreased survival after tumor-inoculation into mice, implying a potential contribution of this receptor to an antitumor immune response (Sugimoto et al (2010) J immunol; Mathieu et al (2012) Immunol. Cell Biol. 82-88; Tsukomo and Yasutomo (2018) Front. Immunol. 9, 1-7).

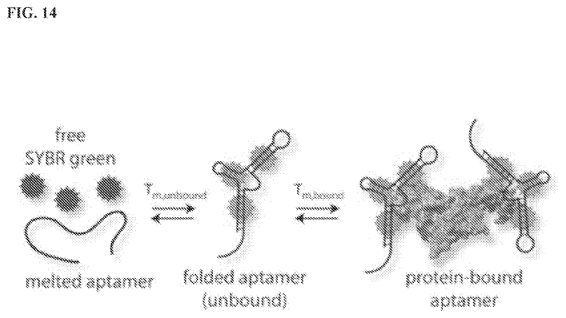

In order to provide direct biochemical evidence that Notch2 is the binding target of CTL3, Thermofluorimetric Analysis (TFA) assay was used. In TFA, DNA-intercalating dyes were used to determine binding constants between DNA-aptamers and target proteins by measuring the temperature-dependent fluorescence of aptamers labeled with SYBR, an intercalating dye, with and without their prospective protein binding partners (Hu, Kim and Easley (2016) HHS Public Access. 7:7358-7362). Upon gradual heating of the aptamer-dye solution, the duplex parts in the aptamer were denatured and the dye was released back to the solution, which highly reduced its fluorescence. Since the aptamer 3D conformation was greatly stabilized upon binding to its respective target protein, the temperature-dependent fluorescence of aptamer-dye complexes varied greatly with and without the putative protein binding partner (FIG. 14).

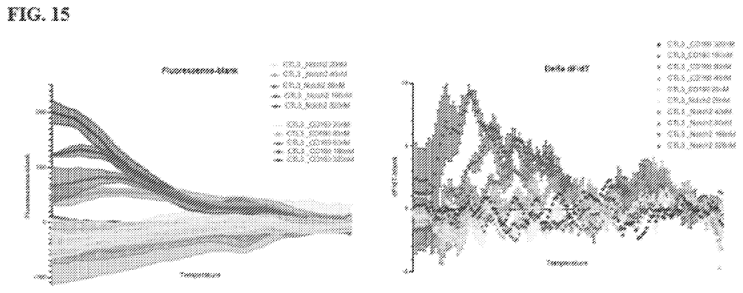

A Tₘ melting curve profile was generated by measuring SYBR green fluorescence during temperature gradient, to monitor aptamer-protein complexes in the presence of different concentrations of either Notch2 or the non-specific control (CD160 protein). Only upon the addition of increasing concentrations of Notch2, and not CD160, a dose-dependent change in CTL3-associated fluorescence was measured (FIG. 15). When looking at the total fluorescence graph, high fluorescence intensity can be seen at 25° C., however, when examining the derivative rate of change of frequency (dF/dT) curves, the temperature-dependent intensity reached a maximum at 37° C.

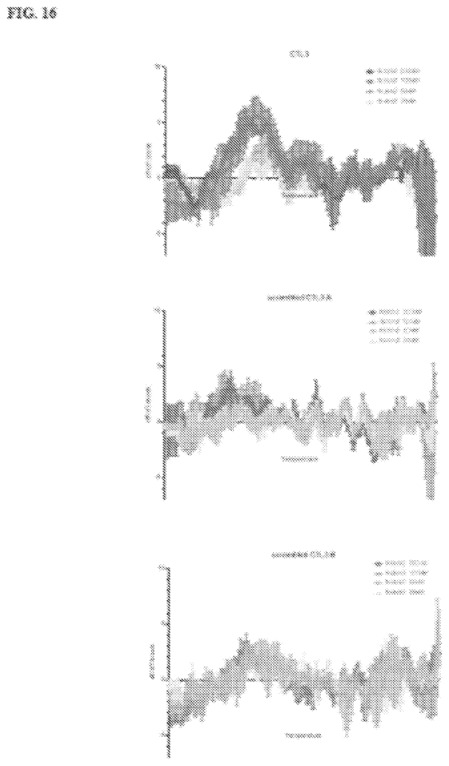

CTL3-Notch2 binding was compared with two scrambled sequences (named scrambled CTL3-A and scrambled CTL3-B) which contain the same base composition. It can be seen from FIG. 16 that CTL3 exhibits a dose-response curve by increasing the concentration of Notch2. This phenomenon is not seen with the scrambled strands, suggesting specific reaction between CTL3 and Notch2 that reaches saturation between 100-200 nM of protein.

In conclusion, in the presence of the DNA intercalating dye, Notch2 protein-bound CTL3 aptamer exhibits a change in fluorescence intensity compared to the intercalated, unbound aptamer. This intensity change does not occur when CD160 is added instead of Notch2, or when scrambled sequences are added.

In contrast to human recombinant Notch2 for which CTL3 aptamer has demonstrated a clear concentration-dependent binding (FIG. 17A), no such pattern was clearly demonstrated for mouse or for rat Notch2, implying less specific binding by CTL3 (FIGS. 17B and 17C).

### Example 4—Materials and Methods for Examples 5-6

A. Materials

a. Random Library

Random library 9.0 (“Lib 9.0”) was purchased from IDT. The library contains a vast repertoire of approximately 10¹⁵ different 40 nt-long random sequences flanked by two 20 nt unique sequences at the 3′ and 5′ acting as a primer for PCR amplification during the SELEX procedure. The lyophilized library was reconstituted in ultra-pure water (UPW) to a final concentration of 1 mM. The random library sequence was: 5′-TCACTATCGGTCCAGACGTA-40N-TATTGCGCCGAGGTTCTTAC-3′ (SEQ ID NO: 54), where N represents a random oligonucleotide selected from a mixture of equally represented T, A, C, and G nucleotides (1:1:1:1 ratio).

Pre-SELEX Preparation:

Following reconstitution, the library underwent QC validation for size exclusion using HPLC ProSEC 300S column (Agilent).

b. Library Primers and Caps

A set of 20 nt primers and caps were purchased from IDT (Table 12). Caps were used to hybridize to the Library's primer sites during incubation with cells in order to refrain from the possibility of primer sequences interacting with the random 40 nt sequence site. A mixture of 3′ and 5′ caps (Table 12) in each SELEX round was used in a 3:1 caps-to-library ratio.

The forward primer was purchased from IDT labelled with Cy-5 at the 5′ site for sequence amplification that was detected in a fluorescence assay. The lyophilized primers were reconstituted in ultra-pure water (UPW) to a concentration of 100 μM.

TABLE 12
Random library, primers and caps sequences

| Auxiliary | SEQ ID | |
|---|---|---|
| sequences | NO: | Sequence 5′ to 3′ |
| Random | 54 | TCACTATCGGTCCAGACGTA-40N- |
| Library | | TATTGCGCCGAGGTTCTTAC |
| Forward | 55 | TCACTATCGGTCCAGACGTA |
| Primer | | |
| Forward | 56 | /Cy5/TCACTATCGGTCCAGA |
| labeled Cy-5 | | CGTA |
| Reverse/ | 57 | GTAAGAACCTCGGCGCAATA |
| 3′ cap | | |
| 5′ cap | 58 | TACGTCTGGACCGATAGTGA |

c. Aptamer Folding Buffer

Phosphate-buffered saline (minus Magnesium and Calcium) was supplemented with 1 mM Magnesium Chloride (MgCl₂). The folding buffer was sterilized with PVDF membrane filter unit 0.22 μm and kept at 4° C.

d. PBMC

PBMC were isolated using Ficoll (Lymphoprep, Axis-Shield) density gradient centrifugation following the manufacturer's protocol.

Frozen Cynomolgus Monkey PBMCs (NHP-PC001) were purchased from Creative Biolabs.

e. Human PanT and B Cell Isolation

Isolation of human Pan T cells was performed by using Pan T cells isolation kit (Miltenyi Biotec, 130-096-535) following the manufacturer's protocol. Isolation of human Pan B cells was performed by using Pan B cells isolation kit (Miltenyi Biotec, 130-101-638) following the manufacturer's protocol

f. Antibodies, Proteins and Enzymes

αCD3ε-FITC (Cat. #130-113-690)/APC (Cat. #130-113-687)/VioBlue (Cat. #130-114-519)/APC-Vio770 (Cat. #130-113-688), αCD4-FITC(Cat. #130-114-531), αCD8-FITC (Cat. #130-113-719)/PE-Vio770 (Cat. #130-113-159) and matching isotype controls were purchased from Miltenyi Biotech. αCD3ε OKT3 clone (Cat. #317302) was purchased from BioLegend.

Recombinant Human CD3 epsilon protein (Fc Chimera His Tag) (ab220590), Recombinant Cynomolgus CD3 epsilon protein (Fc Chimera His Tag) (ab220531), and Recombinant Mouse CD3 epsilon protein (His tag) (ab240841) where purchased from Abcam. Human IgG1 isotype was used as a negative counter selection (InVivoMAb, BE0297).

Protein G magnetic beads purchased from ThermoFisher (88847).

Herculase II Fusion DNA Polymerase (600675) that is used for Assymetric PCR (A-PCR) purchased from Agilent and real-time-PCR iTaq Universal SYBRGreen Supermix (1725124) purchased from BIO-RAD.

g. Cell-Lines

Jurkat, Daudi and Kasumi-1 cell-lines were purchased from ATCC. Jurkat cell (ATCC TIB-152), Daudi cells (ATCC CCL-213) and Kasumi-1 (ATCC CRL-2724) were grown in RPMI-1640 supplemented with 10% fetal calf serum (FCS) and 1% Penicillin and streptomycin (Pen/Strep). All cells were cultured at 37° C. and 5% CO2.

h. Aptamers

Each aptamer was diluted to the desired concentration with the folding buffer. The aptamers were heated for 5 minutes at 95° C., followed by a rapid cooling for 10 minutes on ice, and room temperature (RT) incubation for 10 minutes. Folded aptamer was then added to the medium-suspended cells.

Lyophilized aptamers were kept in dark at RT until reconstituted in PBS-supplemented with 1 mM MgCl₂ to a concentration of 100 μM and stored at −20° C. in the dark.

B. Experimental Methods

a. Binding SELEX Protocol

The binding SELEX was conducted for 11 sequential rounds using CD3ε-Fc protein coupled to protein G magnetic beads (Positive selection), IgG1 protein coupled to protein G magnetic beads or with beads only (Negative selections, starting from round 3 onwards).

i. Beads-Protein Complex Preparation

Magnetic protein G beads were vortexed and washed once with PBS and then mixed with 100 ul of protein for 10 min at RT under gentle shaking condition. Then, the beads were separated by a magnet, the supernatant was discarded and the beads re-suspended with 350 ul of Folding buffer×1 containing 2% BSA.

For verification of the beads-protein complex formation, a small sample (before DNA added) was treated with FC-blocker (Miltenyi), stained with αCD3ε and analysed by flow cytometry

ii. Initial Library and Enriched Round Library Preparation and Folding Protocol

The library is initially reconstituted to 1 mM. Working concentration in the first round was 14.3 μM, while in rounds 2-11, a concentration of 0.25-0.5 μM of enriched library was used. For each round the following components were used:

TABLE 13
Calculating library concentrations

| Component | Concentration | Calculation |
|---|---|---|
| Enriched library | 0.25-0.5 μM | $\begin{matrix}{C}_{μ M} = \frac{{C}_{\frac{ng}{μ l}} \times 1 \text{,} 000}{330 gr / mole \times [ 90 nt ( lib length ) ]} \\ {C}_{μ M} \times {V}_{Elution} = [ 0.2 up to 0.5 ] μM \times {V}_{pool}\end{matrix}$ |
| Mix caps 5’ + 3’ | 50 uM | $\frac{{V}_{Elution} \times {C}_{Elution}}{5 {0}_{μ M}} \times 3 = {V}_{mix caps}$ |
| Folding buffer | X10 | $\frac{{V}_{Elution} + {V}_{mix caps}}{9} = {V}_{FBX 10}$ |
| Folding buffer | X1 | Adjust volume to 350 ul |

The libraries underwent DNA folding per the following protocol: were heated for 5 minutes at 95° C., followed by a rapid cooling for 10 minutes on ice, and maintained until use at 4° C.

iii. SELEX

Once the enriched library was folded, 350 ul of enriched library rounds was added to 350 ul of CD3ε-FC-bead (positive selection rounds 1-11) or to Beads only/IgG₁-beads complex (counter selection, rounds 3-11). Incubation time, protein amount and wash steps varied by the SELEX rounds.

In positive selection, the supernatant, “unbound to positive” fraction, was removed kept at −20° C. until NGS preparation. For washes, the beads were precipitated with a magnet, the supernatant was discarded and the beads were re-suspended with 1 ml of folding buffer×1. After the washing step, the beads suspend in 300 ul ultra-pure water (UPW) and the DNA eluted at 95° C. for 10 min. Finally, the beads precipitated with magnet, and supernatant “bound to positive” was collected for the PCR stage.

If a negative SELEX round was implemented, than the 350 ul of enriched library rounds was added to 350 ul of beads only/IgG beads complex and the supernatant collected fractions proceeded to positive selection stage. The binding fraction to the negative samples, called “bound to negative”, were eluted and kept at −20° C. until NGS preparation.

iv. PCR Amplification Protocol

The eluted DNA fractions (“bound” and “unbound”) were used, each, as a template for Asymmetrical PCR (A-PCR) amplification. The PCR reaction was modulated for each round. The PCR components and the amplification protocol are shown in table 14 and table 15, respectively.

TABLE 14
PCR components

| Reagent | Stock | Volume | |
|---|---|---|---|
| UPW | | Adjust to reaction final | |
| | | volume | |
| Buffer | x5 | x1 | |
| dNTPs mix | 10 μM | 0.8 | mM |
| Forward primer | 10 μM | 2.5 | uM |
| Reverse primer | 10 μM | 0.25 | uM |

| | |
|---|---|
| Template | 15% |
| DNA polymerase | 1% |
| enzyme | |

TABLE 15
PCR amplification protocol for enriched library

| Number of | | | |
|---|---|---|---|
| cycles | Temperature | Duration | |
| 1 | 95° C. | 3 | min |
| 18-36 | 95° C. | 30 | sec |
| | 58° C. | 30 | sec |
| | 72° C. | 30 | sec |

| | | |
|---|---|---|
| Final | 4° C. | ∞ |

v. PCR ssDNA Purification

The PCR products were concentrated with 10K Amicon (Millipore, UFC5010BK) and purified using HLPC ProSEC 300S size exclusion column (Agilent). After purification, the DNA underwent buffer exchange with ssDNA clean kit (ZYMO, D7011), concentration was measured using NanoDrop and the DNA was diluted for a new SELEX round.

b. Assessment of Library Pools Binding to Target Protein by Real-Time-PCR

Magnetic protein G beads were vortexed and washed once with PBS and then re-suspended with protein (CD3ε or IgG₁) for 10 min at RT under gentle shaking condition. Then, the beads were precipitated under the magnetic field, the supernatant was discarded and the beads re-suspended with 125 ul of Folding buffer×1 and 2% BSA. Next, the library pools from rounds 3, 6, 9, 11, and the initial random library were folded (95° C. 5 min, ice 10 min, and maintenance at 4° C.). 125 ul of each of the folded DNA libraries was mixed with the beads-protein complex for 1 hr at 4° C. in a gentle shaking. After incubation, the beads were precipitated with a magnet and washed 3 times with 1 ml folding buffer. Finally, the DNA binding fraction was eluted at 95° C. with 100 ul UPW for 10 min and subsequently used as a template in real-time-PCR with SYBRGreen Supermix (BIO-RAD).

c. Assessment of Individual Aptamers Binding to Target Protein Protein-Aptamers Binding Assay by HPLC

1 μM of folded Cy5 labeled aptamer was mixed with 5 μM of protein to a final volume of 60 ul and incubated for 1 hr at 4° C. or 37° C. Next, to detect the Cy-labelled aptamers, samples were analyzed at 570 nm absorption via HPLC ProSEC 300S size exclusion column (Agilent).

d. Assessment of Individual Aptamers Binding to Cells by with Flow Cytometry

0.5-2×10⁶ cells (isolated Pan T cells, B cells, hPBMCs, Cynomolgus PBMCs, Jurkat, and Daudi) were washed and re-suspended in 0.2-1. ml folding buffer that contains 0.1% BSA and 0.01% tRNA.

0.25-1.25 uM of single DNA candidate were fluorescently labelled by mixing with CpG′-Cy5 tag (1:1 ratio) and folded (95° C. 5 min, ice 10 min, and maintenance at 4° C.). Next, the labelled DNA aptamers were incubated with the cells for 1 hr at 4° C. or 37° C. in V shape 96 well plate under gentle shaking conditions (hPBMCs and Cyno PBMCS were added αCD8/αCD4 in the final 15 min of incubation). After incubation, cells were washed 3 times with folding buffer×1 and analysed after each wash using flow cytometry (CytoFlex).

e. Competitive CD3 Epsilon Epitopes Binding Assay

0.25×10⁶ Jurkat cells were washed once, re-suspended in folding buffer×1 containing 0.1% BSA and 0.01% tRNA and incubated for 15 min with 1:20 dilution of αCD3 clone OKT3 (BioLegend, 317302) or αCD3 clone REA613 (Miltenyi, 130-114-519) or with buffer. Next, 0.25 uM of folded Cy5 labelled aptamers were incubated with the cells for 1 hr at 37° C. under gentle shaking condition. After incubation, cells were washed 3 times with folding buffer×1 and analysed after each wash using flow cytometry (CytoFlex).

f. CS6 Effective Concentration 50 (EC50) Quantification

5×10⁴ Jurkat cells were washed and re-suspended in ×1 folding buffer that contain 0.1% BSA and 0.01% tRNA. 0.1-80 nM of CS6 aptamer were labelled with CpG′-Cy5 tag (1:1 ratio) and folded (950 C 5 min, ice 10 min, and maintenance at 40 C). Next, the DNA aptamers were mixed with the cells and incubated for 1 hr at 370 C in V shape 96 well plate under gentle shaking conditions. After incubation, the cells were washed twice with folding buffer×1 and analysed via flow cytometry (CytoFlex).

### Example 5—Identification of CD3-Targeting Aptamer Via Binding SELEX

A significant optimization step of the drug candidate was carried out via the replacement of the above-mentioned T cell engager with a novel aptamer targeting CD3 epsilon ligand on the surface of T cells.

Selection of the CD3 binding aptamers was described herein. The T cell targeting aptamers were identified via Binding SELEX and Hybrid Binding Cell-SELEX using recombinant CD3e protein and recombinant protein plus T cells, respectively. The final lead was characterized for its binding to the target protein and T-Cells.

This disclosure describes the identification and characterization of the T cell engaging aptamers from a random library of 10¹⁵ potential aptamers using the SELEX methodology in a novel application. This aptamer moiety, as part of the bispecific therapeutic entity was designed to be constant across different patients.

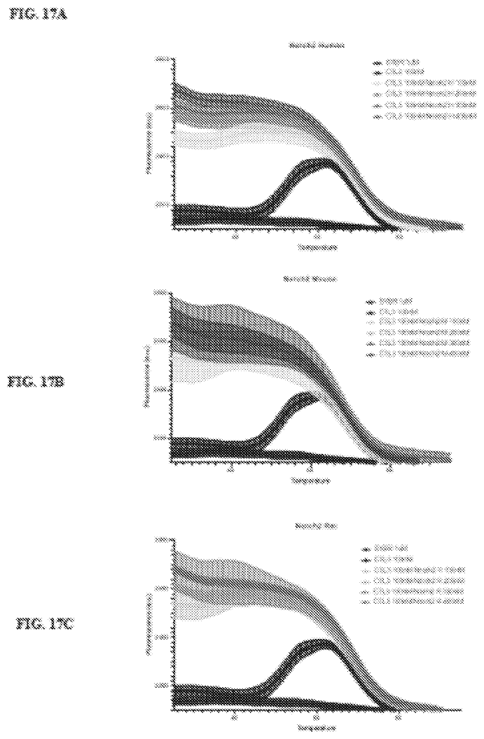

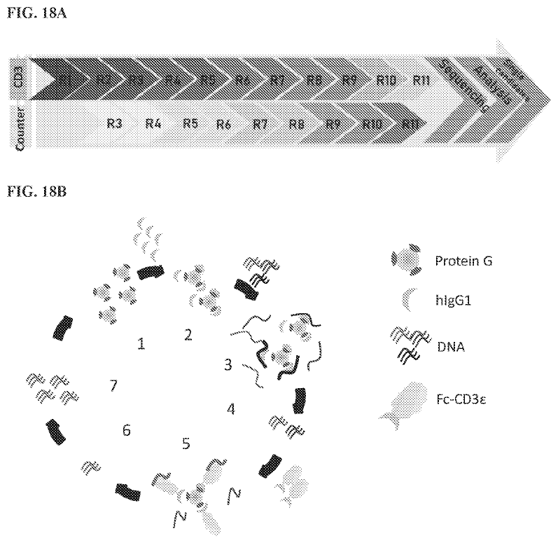

Binding SELEX was conducted using recombinant Human CD3 epsilon protein Fc chimera for a total of eleven (11) rounds. For counter negative selection, either beads only (rounds 1-6) or beads conjugated to Human IgG₁ (rounds 7-11) were used in order to rid of all aptamers which bind non-specifically to the magnetic beads or to the Fc component of the recombinant protein (FIG. 18). After round 11 of the SELEX, enriched aptamer libraries were subjected to sequencing and analysis via specific algorithm. Single candidates were identified and undergo verification.

FIG. 18B depicts the SELEX stages: counter selection starts with protein G magnetic beads (1) that were conjugated to IgG1 (2) and incubated with DNA aptamer library pool from the previous stage (3). Next, unbound DNA aptamers were collected for positive selection (4) and were incubated with FC-CD3ε-conjugated beads (5) here, the bound fraction (6) underwent PCR amplification and HPLC purification for the next round.

1. SELEX Rounds Comparative Assay

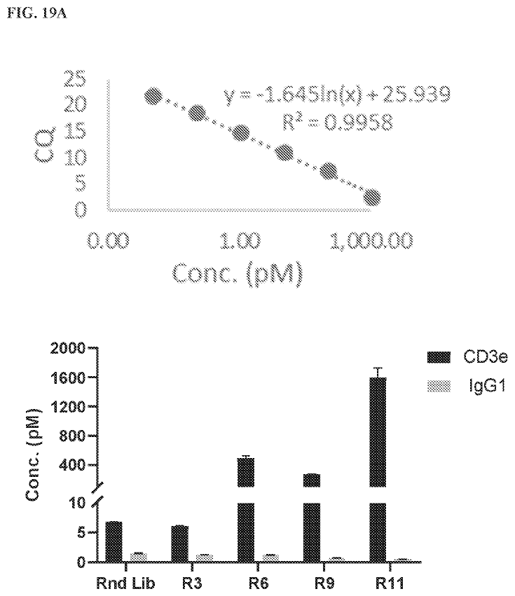

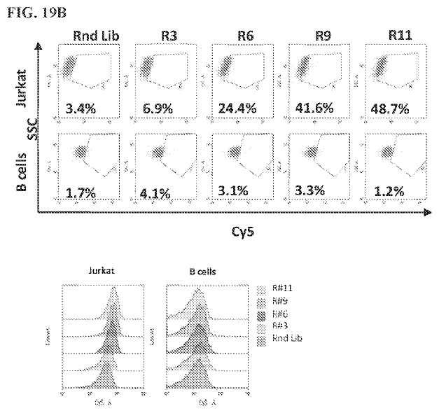

Original random library ‘No. 9.0’ and library pools eluted from rounds 3, 6, 9 and 11 were tested for their binding to hCD3ε. Each round was amplified by PCR using 5′ primer labelled with Cy-5 following incubation with Beads-Fc-CD3ε complex for 1 hr at 4° C. As a negative control, the variant pools where incubate with Beads-IgG1 complex (FIG. 19A). The amount of amplified DNA, which was precipitated with the target protein, was found much higher in libraries from rounds 6, 9 and 11 than in the random initial library used in the binding SELEX. The results showed specific and strong enrichment as of round six compared with the initial library. Further, there was another increment in the specific binding observed in round 11.

After demonstrating round-to-round enrichment using the recombinant CD3 protein, we tested whether such enrichment is observed also in a whole-cell context. Jurkat T cells were incubated with the same Cy5 tagged library pools, washed, and analysed by flow cytometry. As a negative control, isolated Pan B cells were used (FIG. 19B).

Similarly to the protein data, a specific and strong round-to-round enrichment for the target cells was demonstrated.

2. NGS Results

Enriched libraries eluted in rounds 8, 9, 10 and 11 (“bound”), as well as the supernatant of positive selection rounds (“unbound”), were subjected to sequencing using the high-throughput NGS Illumina NextSea500.

Post sequencing, the data was analyzed via an algorithm which allocated single candidates for downstream binding assays. The algorithm utilizes statistical estimators, tests, and metrics.

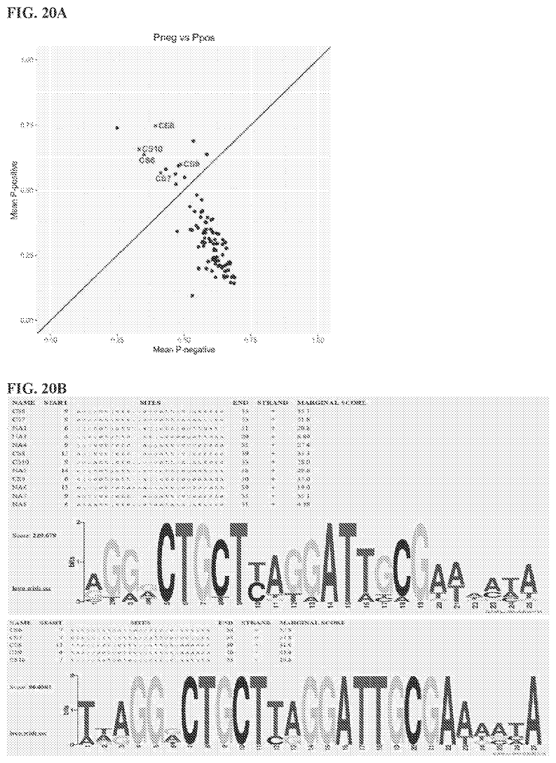

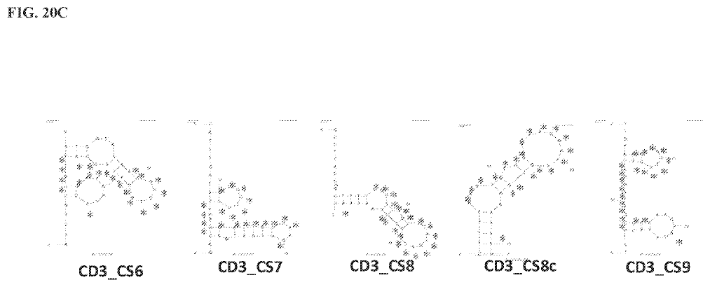

The mean P-positive and P-negative scores of the top 100 most abundant aptamers in the last round, were plotted (FIG. 20A), and aptamers with significant bound to unbound ratio as described above in 46 (p<0.05; Poisson test, consistent in all rounds) were highlighted and selected for experimental validations (termed CD3-CS6-9, ID SEQ NO 88-91). The additional 9 aptamers with high mean P-positive values (P-positive>0.5) were assigned an identifier (CD3_Ppos10-18 ID SEQ NO. 93-101)). The identified CD3 binding aptamers are listed in Table 16

TABLE 16

| CD3-binding aptamers | | |
|---|---|---|
| Aptamer | SEQ ID | |
| name | NO: | Sequence 5′ to 3′ |
| CS6 | 59 | ATCGTATAAGGGCTGCTTAG |
| | | GATTGCGATAATACGGTCAA |
| CS7 | 60 | CATTTCATAGGGCTGCTTAG |
| | | GATTGCGAAGGTAATGCCAG |
| CS8 | 61 | CCCTTACCCCTTTTAGGTCT |
| | | GCTTAGGATTGCGAAAAAAG |
| CS9 | 62 | TTGTAAGGACTGCTTAGGAT |
| | | TGCGAAAACAATATTCGTAT |
| CS8c | 63 | CTTTTAGGTCTGCTTAGGAT |
| | | TGCGAAAAAAG |
| Ppos10 | 64 | TCCATGGGTCTGCTCTAGGA |
| | | TTGCGTTCATGGTCTCCCCG |
| Ppos11 | 65 | AATTACAACCTTGGATTGCA |
| | | AAGGGCTGCTGTGTTGTTTA |
| Ppos12 | 66 | ATCGGAGCTGTTCCTTGATA |
| | | CCGATTCAAAAAGTTCGTAC |
| Ppos13 | 67 | AATTTGTAGGGACTGCTCAG |
| | | GATTGCGGATACAAATTAAT |
| Ppos14 | 68 | AGACATTGGGGACTGCTCGG |
| | | GATTGCGAATCTATGTCTCC |
| Ppos15 | 69 | CCCTTTTTTAACTAGGTCTG |
| | | CTTAGGATTGCGAATGTTAA |
| Ppos16 | 70 | ACCTCAAAAGCGCGGGCTGC |
| | | TCAAAGGATTGCGTAGCTTT |
| Ppos17 | 71 | GGGGGTTAAGGGCTGCTTAG |
| | | GATTGCGATAATACGGTCAA |
| Ppos18 | 72 | AACATATAACTGCTCAATAA |
| | | TATAGATAAAATACTCACAA |

Next, the 14 aptamers with high mean P-positive values (P-positive>0.5) (see Table 16) underwent multiple sequence alignment and a shared motif was found (FIG. 20B upper). In comparison, the highlighted candidates (CS6-9) were also aligned and a more robust motif was discovered (FIG. 20B bottom). In addition, structure prediction analysis was carried by analytic software (mfold, NUPACK) (FIG. 20C). This analysis demonstrated that candidates fold into a complex secondary structure mainly around the motif region. Following this result and in an optimization attempt, CD3_CS8 was further edited by trimming the first 9 nucleotides (denoted CD3_CS8cut) which seemed irrelevant to the formation of the secondary structure around the presented motif in CS_CD8. Top 5 candidates were further confirmed to possess a negative Delta G scores and were selected for individual binding assays.

In addition to the binding SELEX described above, a hybrid methodology was implemented, in which the process included also whole-cell SELEX rounds.

TABLE 17
Alternative CD3-binding aptamers

| Aptamer | SEQ ID | |
|---|---|---|
| name | NO: | Sequence 5′ to 3′ |
| CS1 | 73 | CTCTACCTGACTGTAACCTC |
| | | TCGCTCCCCCCCATTCGCGC |
| CS2 | 74 | TTGTCCCTCTACGCCGCCCT |
| | | TTACTACCACTCCTGCGATT |
| CS3 | 75 | TCCAGCACACCGACCGCCCC |
| | | TCTACATTACCCCCTGGACT |
| CS4 | 76 | CCCCTCCATTCCCCCGCCTC |
| | | GTCCACCCTACTCCTTAGTC |
| CS5 | 77 | CATCGACGCCCACACACCAC |
| | | TTCCCGTTCCCCTGCATCAT |

### Example 6—Individual CD3 Binding Aptamers Validation (of Example 5)

a. Aptamer Candidates Demonstrate Binding to Human CD3ε Via HPLC

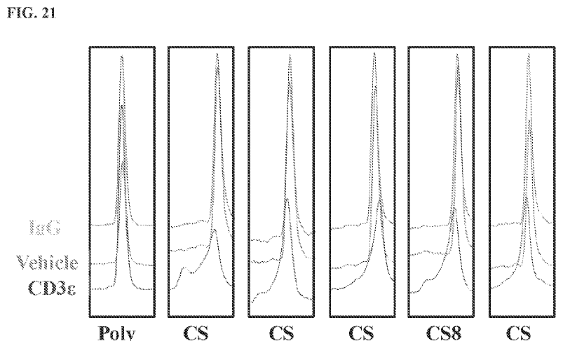

Top five candidates (CS6, CS7, CS8, CS9, and CS8c; SEQ ID NO. 59-63, receptively) were synthesized with a 5(5′) phosphothioated CpG motif and assayed for Human CD3ε (hCD3ε) binding via the HPLC size exclusion column. In this method, the aptamers were labelled with Cy5 complementary sequence to the CpG site (Cy5-CpG′). Then, the folded-labelled candidates are incubated, each, with the CD3ε-recombinant protein or with negative control IgG1 (1 hr at 37° C. and 4° C.) and analyzed by HPLC ProSEC 300S size exclusion column (Agilent) at 570 nm absorption. Upon protein binding, the aptamer-protein complex has a greater mass than a free aptamer and as a result, the retention time (RT) at the column is expected to be shorter. Inversely, in the case of non-binding aptamer, the RT in the presence of protein will be the same as in the absence of the protein. As a control, PolyT sequence was used. All five candidates demonstrated a binding to CD3 epsilon target protein at varying levels (FIG. 21)

b. Aptamer Candidates Demonstrate Specific Binding to Jurkat T Cell Line and Primary Human Pan T Cell by Flow Cytometry

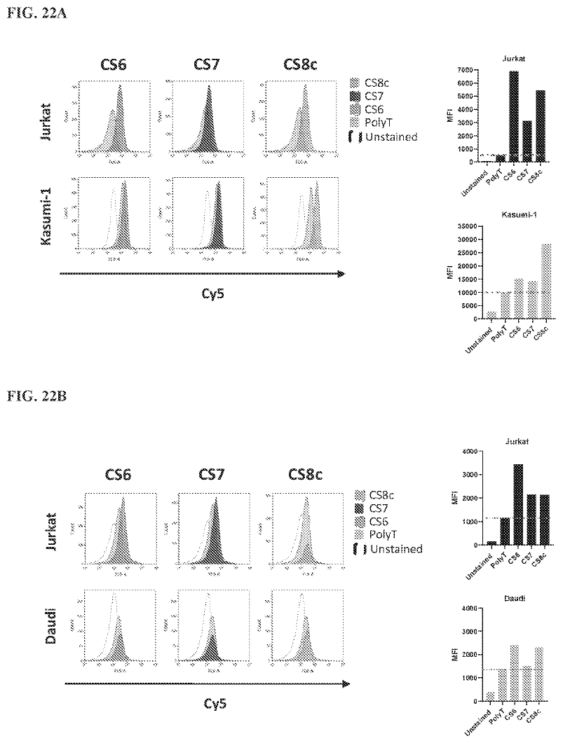

After CS6, CS7 and CS8c candidates demonstrated specific binding to CD3e recombinant protein, they were assayed for binding to their target in the native, whole-cell context, on the surface of T cells by flow cytometry. For this purpose, Jurkat T lymphocyte cell line (Acute T cell leukemia, ATCC TIB-152), previously reported to exhibit TCR expression, were used. The first binding assay with cells conducted at 4° C. for 1 hr. As a negative control, the myeloblast Kasumi-1 cell line was used (Acute myeloblastic leukemia, ATCC CRL-2724) All three candidates were found to differentially bind the target cells as compared with control cells while CS6 and CS7 demonstrated better specificity than CS8c. (FIG. 22A)

Next, to better mimic physiological conditions, the three candidates were assayed for binding Jurkat at 37° C. Here, as a negative control, B lymphoblast Daudi cell line was used (lymphoblast, ATCC CCL-213) (FIG. 22B). In this experiment, the three candidates bound the target cells when CS6 showed the highest binding level.

CS6 was selected for further exploring and characterization. It was found to bind normal primary Pan T cells and not Pan B cells at 37° C. under blocking conditions (FIG. 22C).

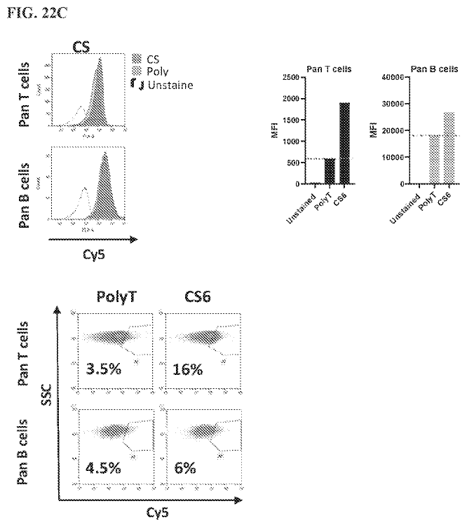

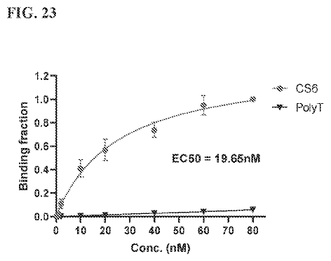

Subsequently CS6 effective concentration 50 (EC₅₀) was evaluated. A serial dilution of -Cy5 labelled aptamer was incubated with Jurkat cells for 1 hr at 37° C. and assessed for binding via flow cytometry (FIG. 23). The calculated EC₅₀ value was 19.65 nM.

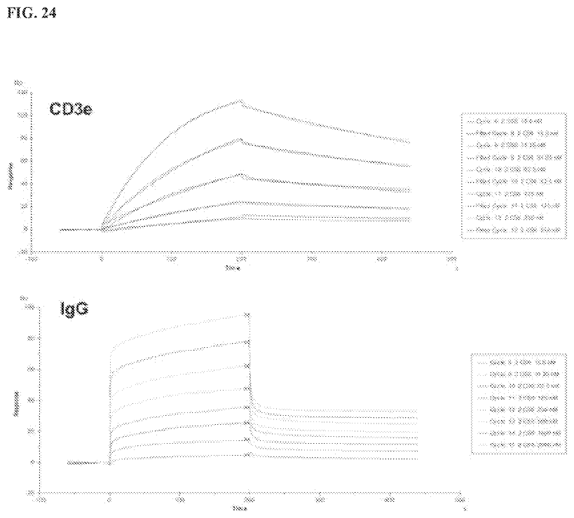

Further, CS6 affinity towards CD3ε was tested by surface plasmon resonance (SPR) and its dissociation constant was calculated to be K_{d}=31 nM (FIG. 24).

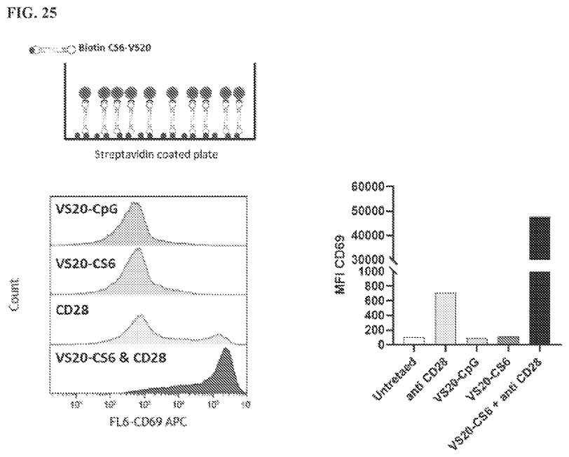

When hybridized to a Variable Strand exemplary sequence VS20 (SEQ ID NO: 78) to form a bispecific T cell engager structure, CS6 has led to the stimulation of T cells, as demonstrated by elevation of CD69 markers (FIG. 25).

### Example 7—Material and Methods for Example 8

A. Materials

a. Aptamers

Cancer-targeting aptamer arm, Variable Strand 12 (VS12, SEQ ID NO: 40) was derived from a functional enrichment process as described in PCT Application No. PCT/IB19/01082 using HCT-116 colon carcinoma cell line as target cells. T cell engager sequence (CTL3 SEQ ID NO: 3) was derived from Cell-SELEX binding process as described in Examples 2-3. Aptamers were synthesized as one oligonucleotide chain and purified using the standard desalting method or were column purified. Complementary CpG-motif sequences were added to both cancer-targeting and immune engager aptamers to allow hybridization and the generation of bispecific aptamer conjugate. Full length sequences are found in Table 18.

TABLE 18
T cell engager sequences, with exemplary hybridization motives to generate
aptamer conjugates, putative modifications and cancer-targeting aptamers

| | | SEQ | |
|---|---|---|---|
| | Aptamer | ID | |
| Category | name | NO | Sequence 5′ to 3′ |
| T cell | CTL1 | 1 | TACGCGCAATTCGCCTTGTCGGTGATCTTCCTTTGAACTTGGGCAGTCT |
| engager | | | G |
| | CTL2 | 2 | TGGCCTGGCCGTGTCGTCTGCTTTATAGTCGGTGATCCCTTGTGTTAATT |
| | CTL3 | 3 | GCATACCTTTCGTATGCCTTTTTGACCCGTATTTTTGCCCTACCCTTCGG |
| | CTL4 | 4 | TTTTTCGCTATCCAACCCTTCTTTCCAGCCTGCCAATCAGTCGGTGATC |
| | | | A |
| | CTL5 | 5 | AGGGCAGTCCTGTATCTTAACATTCTCCTACATCCGTAAGTCGGTGATC |
| | | | C |
| | CTL6 | 6 | GGGCTAAGAGTCTCTATTGTCGGCAGTCGTCTAATATTTCCCGTCCAAT |
| | | | T |
| | CTL7 | 7 | ACTTCCGGTGATTTGATTTCACTTCCTGGGCAGTCAATGTGATTCTCTA |
| | | | C |
| | CTL8 | 8 | GGTCAGTCGCCTTTGTCGGTGATGTACTCGCGCAGTCGGGTTCCCCTTA |
| | | | A |
| | CTL9 | 9 | GGGTCTGTTGGTCCTAGGGCAGTCGTACTTCTAATTCTTGTCCCGATGA |
| | | | T |
| | CTL10 | 10 | CTTGTCGGTGATCTATAGTCGGTGATATATTTTGTCCTATGGTAGTCGA |
| | | | T |
| | CTL11 | 11 | GGGCTCATGGGCAGTCTTTTTACTACCTCCTATTTACGTATCCCGCTCCT |
| | CTL12 | 12 | CACCCGCGCATTTCCCCCCAGTCGGTGATTCTTATATGTACCTGTTCCT |
| | | | C |
| | CTL13 | 13 | GGGCACGTCCATTCGCGTTTTTGTTCCGTTTCTCCCTTTTTGGATTTTGC |
| | CTL14 | 14 | CAGTCGGTGTCACTCCAGCGGTCGGTTCACTCCACATTCTCCCATCTGT |
| | | | C |
| | CTL15 | 15 | GGCAGTCACCATTCTCTTTGGGCAGATTGTCTCTCATCCATATGTCTCC |
| | | | T |
| | CTL16 | 16 | CTACCTCCTTAGTCGGTGATTCGATCTATGGGCCTAACTGCCTTCTCTG |
| | | | T |
| | CTL17 | 17 | GGGATGCGGGGCCCCGTTCTTTTTGTCTCTCATTTTGTCACTTTTTTTGT |
| | CTL18 | 18 | GGTCAGTCCCTTCGGCATGTCGGGATTCCCTCTTTTCGCCTCGTTTCTTT |
| | CTL19 | 19 | GGCTGTCGAACTTTCTCCCTCCCACCGCAGTCGGCCCCTCATCAGTCGT |
| | | | A |
| | CTL20 | 20 | ACTTCCGGTGATTTGATTTCACTTCCTGGGCAGTCAATGTGATTCTCTA |
| | | | C |
| | CTL21 | 21 | ACGTCTGTCGGTGACCTGTAATAGTTTATGTCGGTGATACAGCTTTCCC |
| | | | T |
| | CTL22 | 22 | CTGTCGGTGATCATATAACGCAGTCGGTGTAGTTTAATCCCACTCCCCT |
| | | | A |
| | CTL23 | 23 | GGCCAGTGTCCCAGTCGTGATTGTAATATTAGATTCTTTGTGGCAGTCG |
| | | | T |
| | CTL24 | 24 | ACTCGTCGGTGATTTTAGACCTTTCTCGGTGATCAACACGTCATGCTAT |
| | | | T |
| | CTL25 | 25 | GCCTCGATATCCTCAGGAGTCGGTGTTTCATTCAATCGTCGGTGATAAA |
| | | | T |
| | CTL26 | 26 | GGTCAGTCCGTATACCGCCAATCCGAACCGCAGTCGGTGTCCGCTTTTA |
| | | | C |
| | CTL27 | 27 | TCGGGTTAGATGTCGGTCCCACTATATGTCGGTGATCTAATATTGAACT |
| | | | T |
| CpG motif | CpG1\|CTL3 | 28 | TCGTCGTCGCGGTTCGCGTCCGTGCATACCTTTCGTATGCCTTTTTGACC |
| -T cell | | | CGTATTTTTGCCCTACCCTTCGG |
| Engager | | | |
| strand | | | |
| | 5PS- | 29 | T*C*G*T*C*GTCGCGGTTCGCGTCCGTGCATACCTTTCGTATGCCTTTTT |
| | CpG1\|CTL3 | | GACCCGTATTTTTGCCCTACCCTTCGG |
| | 10PS- | 30 | T*C*G*T*C*GTCGCGGTTCGCG*T*C*C*G*TGCATACCTTTCGTATGCCT |
| | CpG1\|CTL3 | | TTTTGACCCGTATTTTTGCCCTACCCTTCGG |
| | FullPS- | 31 | T*C*G*T*C*G*T*C*G*C*G*G*T*T*C*G*C*G*T*C*C*G*TGCATAC |
| | CpG1\|CTL3 | | CTTTCGTATGCCTTTTTGACCCGTATTTTTGCCCTACCCTTCGG |
| | CpG1\|CTL6 | 32 | TCGTCGTCGCGGTTCGCGTCCGTGGGCTAAGAGTCTCTATTGTCGGCAG |
| | | | TCGTCTAATATTTCCCGTCCAATT |
| | CpG1 CTL5 | 33 | TCGTCGTCGCGGTTCGCGTCCGTAGGGCAGTCCTGTATCTTAACATTCT |
| | | | CCTACATCCGTAAGTCGGTGATCC |
| | CpG1-CS6 | 80 | TCGTCGTCGCGGTTCGCGTCCGTATCGTATAAGGGCTGCTTAGGATTGC |
| | | | GATAATACGGTCAA |
| Non-CpG | Non- | 34 | CTTAATCAGACATTATACAAATTGCATACCTTTCGTATGCCTTTTTGAC |
| 22b | CpG\|CTL3 | | CCGTATTTTTGCCCTACCCTTCGG |
| complementary | | | |
| seq-T | | | |
| cell | | | |
| engager | | | |
| strand | | | |
| | Non- | 35 | CTTAATCAGACATTATACAAATTGGGCTAAGAGTCTCTATTGTCGGCAG |
| | CpG\|CTL6 | | TCGTCTAATATTTCCCGTCCAATT |
| | Non-CpG | 36 | CTTAATCAGACATTATACAAATTAGGGCAGTCCTGTATCTTAACATTCT |
| | CTL5 | | CCTACATCCGTAAGTCGGTGATCC |
| Non-CpG | Non CpG | 37 | GAATTAACAATTATAACGTTTGCATACCTTTCGTATGCCTTTTTGACCC |
| 18b | 18b\|CTL3 | | GTATTTTTGCCCTACCCTTCGG |
| complementary | | | |
| seq-T | | | |
| cell | | | |
| engagers | | | |
| | Non CpG | 38 | GAATTAACAATTATAACGTTTAGGGCAGTCCTGTATCTTAACATTCTCC |
| | 18b\|CTL5 | | TACATCCGTAAGTCGGTGATCC |
| | Non CpG | 39 | GAATTAACAATTATAACGTGGGCTAAGAGTCTCTATTGTCGGCAGTCGT |
| | 18b\|CTL6 | | CTAATATTTCCCGTCCAATT |
| Cancer | HCT116- | 40 | GATTGATCTATTTTCCATATCGCGTTGAGTGTAAAGCCACGAAGGGTTA |
| cell- | VS12 | | T |
| targeting | | | |
| variable | | | |
| strands | | | |
| | CpG1′\|HCT11 | 41 | CGGACGCGAACCGCGACGACGATGATTGATCTATTTTCCATATCGCGTT |
| | 6-VS12 | | GAGTGTAAAGCCACGAAGGGTTAT |
| | 5PS- | 42 | C*G*G*A*C*GCGAACCGCGACGACGATGATTGATCTATTTTCCATATCG |
| | CpG1′\|HCT11 | | CGTTGAGTGTAAAGCCACGAAGGGTTAT |
| | 6-VS12 | | |
| CpG | CpG1′\|A549- | 78 | C*G*G*A*C*GCGAACCGCGACGACGATAGCAATCATAT |
| motif- | VS20 | | GGCTGTGCTCATTTAATAAGCAAGCTGGG |
| Variable | | | |
| Strand | | | |
| | CpG1′\|4T1- | 79 | CGGACGCGAACCGCGACGACGATAAACTCTATCGTCCA |
| | VS32 | | GAGAGAATGTCTGCCTACTGATTTG |

b. Cell Lines and PBMCs Isolation

HCT-116 human colorectal cell line (ATCC® CCL-247™) were cultured in McCoy's 5A supplemented with 10% fetal calf serum (FCS) and 1% Penicillin and streptomycin (Pen/Strep).

PBMCs were isolated by Ficoll density gradient centrifugation from peripheral blood from healthy donors (MIDA Israel, Sheba hospital) using Lymphoprep™ (Axis-Shield) following the manufacturer's protocol. Isolated PBMCs were maintained in RPM11640 from ATCC and supplemented with 1000 fetal calf serum (FCS) and 1% Penicillin and streptomycin (Pen/Strep).

c. Formulation Buffer/Vehicle

Phosphate-buffered saline (minus Magnesium and Calcium) supplemented with 1 mM Magnesium Chloride (MgCl₂). The folding buffer is sterilized with PVDF membrane filter unit 0.22 μm and kept at RT.

B. Experimental Methods

a. Bispecific Personalized Aptamer Formulation

Formulation procedure includes the following steps:

1. Reconstitution

Each strand is diluted/reconstituted (if lyophilized) to the desired concentration in the formulation buffer.

2. Aptamer Folding:
a. Strands are heated for 5 minutes at 95° C.
b. Rapid cooling for 10 minutes on ice.
c. Incubation for 10 minutes at RT.

3. Bispecific Entity Formation

The two strands (cancer-targeting variable strand and the immune engager strand) are then mixed together and incubated in a rotator for 30 minutes at RT.

b. Animals

Female NSG mice, 7-8 week old, were purchased from Jackson Labs. All animal procedures were performed in the facilities of Tel Aviv Sourasky medical center under ethical approval.

c. Xenograft Models Induction and Interventions

HCT116 Early Intervention Model

Female NSG mice were injected subcutaneous (SC) into the mouse right flank with 2×10⁶ HCT116 tumor cells admixed with 0.5×106 fresh human PBMC in a 1:4 ratio with Cultrex® (Basement Membrane Matrix, Type 3), 0.2 ml/mouse. Regimen of SC interventions is detailed per experiment.

d. Tumor Volume Method of Evaluation

Change in tumor volume was monitored by calipers three times per week. Tumor volume was estimated as follows: Tumor Volume (mm3)=length×width²/2

e. Statistical Methods

All quantitative data are expressed as the mean±SEM. Either ANOVA or Student t-test were used, when appropriate, in order to evaluate significance of difference between groups.

### Example 8—Proof-of-Concept (POC) for Aptamer Bispecific Conjugate Efficacy

A. Representative Structures of Bispecific Conjugate Aptamers

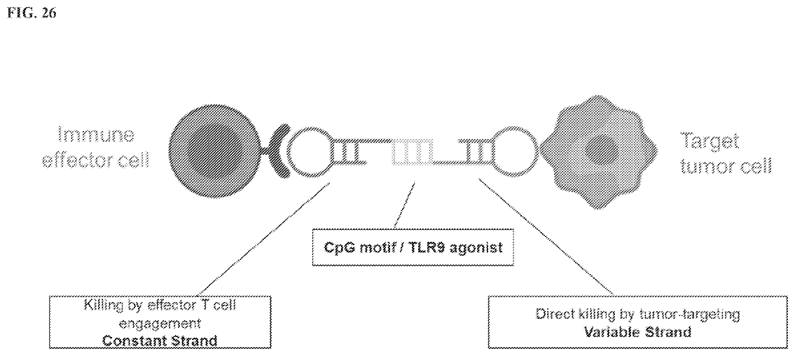

In some aspects, personalized cancer therapeutics described herein are composed of a heterodimeric structure with three separate domains (FIG. 26).

In some embodiments, bispecific personalized, conjugated aptamers are designed to target specific neoantigens and surface molecules displayed by cancer cells of patients and to facilitate both direct lethality of cancer cells as well as immune-associated responses. In some embodiments, efficacy is achieved through three separate modes-of-actions (MoAs) incorporated into a single therapeutic entity, as described below and depicted in FIG. 27:

1. Personalized Strand: Direct Killing of Cancer Cells by Personalized Aptamer

In some embodiments, this moiety is selected through a process initiating from a random pool of 10¹⁵ potential leads and is described in detail in the PCT Application No. PCT/IB19/01082. Briefly, the personalized process is designed to identify aptamers that best facilitate targeted killing of cancer cells while not harming healthy cells. The patient-specific strand is identified by conducting Binding and Functional Enrichment Processes (Cell and Functional SELEX), screening candidates with high-throughput microscopy, and confirming the activity and specificity of top candidates, while including selectivity tests and attempting to rule out off-target effects.

2. Immune-Modulating Strand: Cancer Cell Lysis Through T Cell-Mediated Cytotoxicity

In some embodiments, this aptamer arm is immune effector-targeting and designed to mediate target cancer cell lysis through engaging either target cytotoxic T cells (CTL).

3. CpG Motif with TLR9-Agonistic Activity

The two aptamer arms of the bispecific structure are bridged together by nucleic-base hybridization of single stranded overhangs of complementary sequences. This hybridization domain is CpG rich and designed to induce TLR9-mediated antigen presenting cell (APCs) stimulation and increased uptake of tumor antigens. Stimulated APCs would subsequently migrate to the tumor draining lymph nodes and cross-present the engulfed tumor antigens to cytotoxic T lymphocytes, resulting in an adaptive, systemic, anti-tumor immune response.

B. In Vitro POC of CD3-Targeting Bispecific Aptamer Conjugate

VS12 was hybridized to the T cell engager moiety (the CS) to form the bispecific, dual-acting aptamer CS6-VS12. CS6-VS12 Bispecific Aptamer was assessed for its ability to induce target cell cytotoxicity.

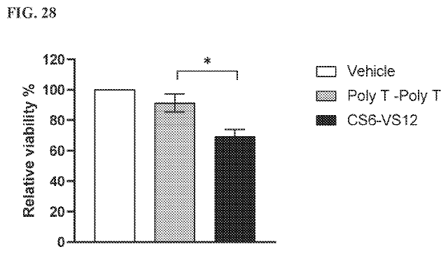

CS6-VS12 was tested for a cytotoxic effect on the HCT116 colon carcinoma cell line in a co-culture setting containing effector PBMCs from healthy donors in an Effector-to-Target (E:T) ratio of 10:1. Tumor cell viability was subsequently analyzed by luminescence-based cell viability assay. CS6-VS12 was compared with the Vehicle negative control (1×PBS supplemented with 1 mM MgCl₂) and a non-specific DNA dimer comprised of two poly-thimidine (PolyT) arms, each of similar oligomer length as the bispecific strands (FIG. 28).

C. In Vivo POC of Bispecific Aptamer Conjugate in HCT116 Tumor Xenograft Model

HCT116 colon carcinoma cells were co-implanted with fresh human PBMC from healthy donors in an immune-deficient female NOD scid gamma (NSG) mice, followed by administration of Vehicle, PolyT conjugate or CTL3 T cell engager (SEQ ID NO: 28) conjugated with VS12 cancer-targeting aptamer (SEQ ID NO: 41) to yield CTL3∥VS12. Study intervention regimen is detailed in Table 19.

TABLE 19
in vivo treatment schedule

| | Dose | Route of | Number of | |
|---|---|---|---|---|
| Treatment | (mg/kg) | Administration | interventions | Days of treatment |
| Untreated | N/A | N/A | N/A | N/A |
| Vehicle | N/A | SC | 10 | 0, 1, 2, 3, 4, 6, 7, 8, 9, 10 |
| PolyT\| \|PolyT | 100 | SC | 10 | 0, 1, 2, 3, 4, 6, 7, 8, 9, 10 |
| CTL3\| \|VS12 | 100 | SC | 10 | 0, 1, 2, 3, 4, 6, 7, 8, 9, 10 |

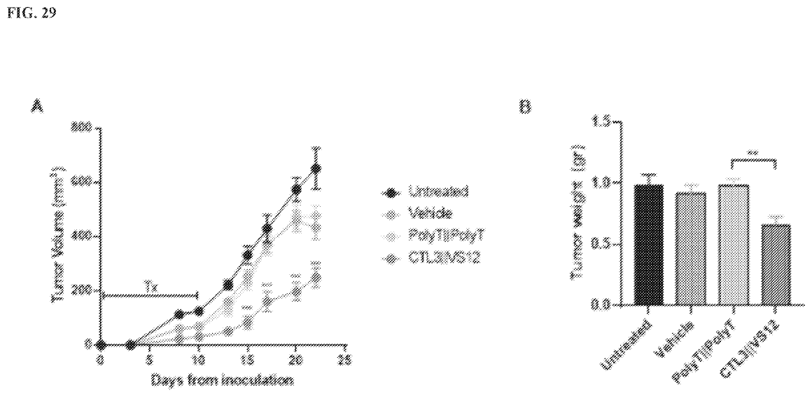

FIGS. 29A and 29B describe HCT116 tumor growth kinetics. Treatment with the Bispecific Aptamer CTL3∥VS12 but not with the non-specific PolyT∥PolyT dimer, has significantly attenuated the growth of HCT116 tumors (FIG. 29A), resulting in an average tumor size which is approximately 30% smaller, in weight, than the control groups on Day 22 (FIG. 29B).

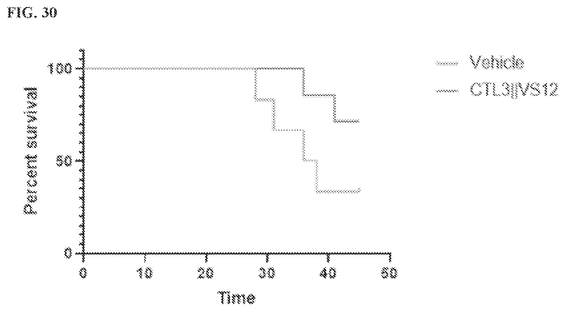

Further, CTL3∥VS12-associated tumor growth attenuation has conferred a better survival rate (FIG. 30).

D. CS6-VS12 Bispecific Aptamer Attenuates Tumor Growth In Vivo

In the xenograft model, HCT116 colon carcinoma cells were co-implanted with fresh human PBMC from healthy donors in an admix manner (E:T 1:4 ratio), in immune-deficient female NSG mice, and were administered with Bispecific Personalized Aptamer (CS6-VS12, SEQ ID NOs: 59 and 42), PolyT duplex, or vehicle.

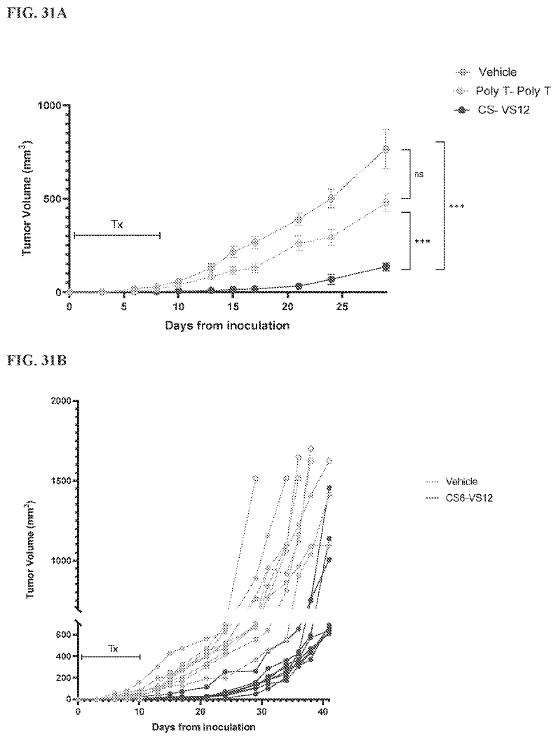

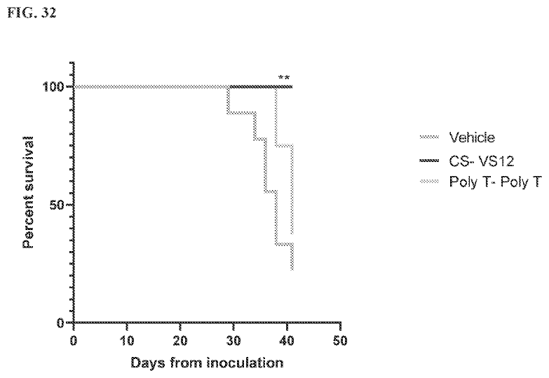

FIGS. 31A and 31B describe HCT116 tumor growth kinetics. Treatment with the Bispecific aptamer CS6-VS12, but not with the non-specific oligonucleotide PolyT, significantly attenuated the growth of HCT116 tumors after a total of 10 interventions. As of Day 30, mice began to be scarified due to ethical volume for endpoint. Individual mice tumor volume were presented until Day 41 (31 days after last intervention). Inhibition in tumor growth was demonstrated in all CS6-VS12 treated mice (FIG. 31B). Tumor growth reduction was translated to a benefit in survival for the bispecific-treated group, as compared to Vehicle (FIG. 32).

E. Murine 4T1-Targeting Bispecific Aptamer CS6-VS32 Efficacy In Vivo, in Combination with Immune Checkpoint Inhibitor

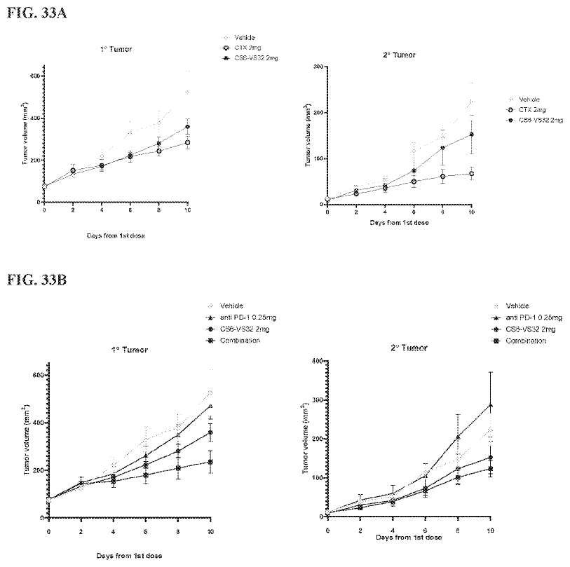

In order to enable efficacy in vivo animals in immunocompetent animals (in addition to the above-mentioned xenograft models), the murine breast cancer cell line 4T1 was subjected to the functional enrichment platform (similarly to other examples in Example 10) and VS32 was identified. VS32 was hybridized to CS6 to form the bispecific aptamer and was assessed in a dual-flank 4T1 tumor model. A trend of hindered growth of both the primary and secondary tumors was demonstrated by intratumoral administration of CS6-VS32 into the primary established tumor (FIG. 33A). Cyclophosphamide (CTX) chemotherapy was used as a positive control, in an equivalent dose. When administration of CS6-VS32 was combined with the immune checkpoint inhibitor anti-PD1, a synergistic effect was demonstrated, leading to a significant tumor growth reduction, both at the injected tumor and in the secondary, non-injected one (FIG. 33B).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. An aptamer comprising a nucleic acid sequence that is at least 80% identical to any one of SEQ ID NOs: 1-39, 59-77 or 80.

2. The aptamer of claim 1, wherein the aptamer comprises a nucleic acid sequence that is at least 90% identical to any one of SEQ ID NOs: 1-39, 59-77 or 80.

3. The aptamer of claim 1 or 2, wherein the aptamer comprises a nucleic acid sequence that is at least 95% identical to any one of SEQ ID NOs: 1-39, 59-77 or 80.

4. The aptamer of any one of claims 1 to 3, wherein the aptamer comprises a nucleic acid sequence that is at least 98% identical to any one of SEQ ID NOs: 1-39, 59-77 or 80.

5. The aptamer of any one of claims 1 to 4, wherein the aptamer comprises a nucleic acid sequence of any one of SEQ ID NOs: 1-39, 59-77 or 80.

6. The aptamer of any one of claims 1 to 4, wherein the aptamer comprises a nucleic acid sequence of any one of SEQ ID NOs: 3, 5, 6, 28, 59, 80, and 29.

7. An aptamer comprising at least 20 consecutive nucleotides of any one of SEQ ID NO: 1-39, 59-77 or 80.

8. The aptamer of claim 7, wherein the aptamer comprises at least 30 consecutive nucleotides of any one of SEQ ID NO: 1-39, 59-77 or 80.

9. The aptamer of claim 7 or 8, wherein the aptamer comprises at least 40 consecutive nucleotides of any one of SEQ ID NO: 1-39, 59-77 or 80.

10. The aptamer of any one of claims 7 to 9, wherein the aptamer comprises at least 50 consecutive nucleotides of any one of SEQ ID NO: 1-39, 59-77 or 80.

11. The aptamer of any one of claims 7 to 10, wherein the aptamer comprises at least 50 consecutive nucleotides of any one of SEQ ID NOs: 3, 5, and 6, at least 40 consecutive nucleotides of SEQ ID NO: 59, at least 63 consecutive nucleotides of SEQ ID NO: 80, or comprises at least 73 consecutive nucleotides of SEQ ID NO: 28 or 29.

12. An aptamer of any one of claims 1 to 11, wherein the aptamer is no more than 100 nucleotides in length.

13. The aptamer of any one of claims 1 to 12 wherein the aptamer is no more than 90 nucleotides in length.

14. The aptamer of any one of claims 1 to 13, wherein the aptamer is no more than 80 nucleotides in length.

15. The aptamer of any one of claims 1 to 14, wherein the aptamer is no more than 73 nucleotides in length.

16. The aptamer of any one of claims 1 to 15, wherein the aptamer binds to a T cell.

17. The aptamer of any one of claims 1 to 16, wherein the aptamer binds to a CD8+ cytotoxic T cell.

18. The aptamer of any one of claims 1 to 17, wherein the aptamer binds to a T cell antigen selected from Notch 2 and other Notch family members, KCNK17, CD3, CD28, 4-1BB, CTLA-4, ICOS, CD40L, PD-1, OX40, LFA-1, CD27 PARP16, IGSF9, SLC15A3 and WRB.

19. The aptamer of any one of claims 1 to 18, wherein the aptamer induces: (a) T cell-mediated cytotoxicity; (b) cell death of a cancer cell through T cell-mediated cytotoxicity.

20. The aptamer of any one of claims 1 to 19, wherein the aptamer induces: (a) cytokine secretion; and/or (b) T cell activation.

21. The aptamer of claim 20, wherein the aptamer induces cell death of a cancer cell in vitro.

22. The aptamer of claim 20, wherein the aptamer induces cell death of a cancer cell in vivo.

23. The aptamer of any one of claims 20 to 22, wherein the cell death is apoptosis.

24. The aptamer of any one of claims 20 to 23, wherein the cancer cell is a patient-derived cancer cell.

25. The aptamer of any one of claims 20 to 24, wherein the cancer cell is a solid tumor cell.

26. The aptamer of claim 25, wherein the cancer cell is a breast cancer cell or a colorectal carcinoma cell.

27. An aptamer of any one of claims 1 to 26, wherein the aptamer comprises a chemical modification.

28. The aptamer of claim 27, wherein the aptamer is chemically modified with poly-ethylene glycol (PEG).

29. The aptamer of claim 28, wherein the PEG is attached to the 5′ end of the aptamer.

30. The aptamer of any one of claims 27 to 29, wherein the aptamer comprises a 5′ end cap.

31. The aptamer of any one of claims 27 to 30, wherein the aptamer comprises a 3′ end cap.

32. The aptamer of claim 31, wherein the 3′ end cap is an inverted thymidine.

33. The aptamer of claim 31, wherein the 3′ end cap comprises biotin.

34. The aptamer of any one of claims 27 to 33, wherein the aptamer comprises a 2′ sugar substitution.

35. The aptamer of claim 34, wherein the 2′ sugar substitution is a 2′-fluoro, a 2′-amino, or a 2′-O-methyl substitution.

36. The aptamer of any one of claims 27 to 35, wherein the aptamer comprises a locked nucleic acid (LNA), unlocked nucleic acid (UNA) and/or 2′deozy-2′fluoro-D-arabinonucleic acid (2′-F ANA) sugars in its backbone.

37. The aptamer of any one of claims 27 to 36, comprises a methylphosphonate internucleotide bond and/or a phosphorothioate (PS) internucleotide bond.

38. The aptamer of any one of claims 27 to 37, wherein the aptamer comprises a triazole internucleotide bond.

39. The aptamer of any one of claims 27 to 38, wherein the aptamer is modified with a cholesterol or a dialkyl lipid.

40. The aptamer of claim 39, wherein the cholesterol or dialkyl lipid is linked to the 5′ end of the aptamer.

41. The aptamer of any one of claims 27 to 40, wherein the aptamer comprises a modified base.

42. The aptamer of any one of claims 1 to 41, wherein the aptamer is a DNA aptamer.

43. The aptamer of claim 42, wherein the aptamer is a D-DNA aptamer.

44. The aptamer of claim 42, wherein the aptamer is an enantiomer L-DNA aptamer.

45. The aptamer of any one of claims 1 to 41, wherein the aptamer is an RNA aptamer.

46. The aptamer of claim 45, wherein the aptamer is a D-RNA aptamer.

47. The aptamer of claim 45, wherein the aptamer is an enantiomer L-RNA aptamer.

48. An aptamer conjugate comprising an aptamer of any one of claims 1 to 47 is linked to a cancer cell-binding moiety.

49. The aptamer conjugate of claim 48, wherein the aptamer is covalently linked to the cancer cell-binding moiety.

50. The aptamer conjugate of claim 48, wherein the aptamer is non-covalently linked to the cancer cell-binding moiety.

51. The aptamer conjugate of any one of claims 48 to 50, wherein the aptamer is directly linked to the cancer cell-binding moiety.

52. The aptamer conjugate of any one of claims 48 to 50, wherein the aptamer is linked to the cancer cell-binding moiety via a linker.

53. The aptamer conjugate of any one of claims 48 to 52, wherein the cancer-cell binding moiety binds to an antigen expressed on a cancer cell

54. The aptamer conjugate of any one of claims 48 to 53, wherein the cancer-cell binding moiety induces cell death when contacted to a cancer cell.

55. The aptamer conjugate of any one of claims 48 to 54, wherein the cell death is apoptosis.

56. The aptamer conjugate of any one of claims 48 to 55, wherein the cancer cell is a solid tumor cell.

57. The aptamer conjugate of claim 56, wherein the cancer cell is a breast cancer cell or a colorectal carcinoma cell.

58. The aptamer conjugate of any one of claims 48 to 57, wherein the cancer-cell binding moiety induces cell death when contacted to the cancer cell in vitro.

59. The aptamer conjugate of any one of claims 48 to 58, wherein the cancer-cell binding moiety induces cell death when contacted to the cancer cell in vivo.

60. The aptamer conjugate of any one of claims 48 to 59, wherein the cancer cell-binding moiety is an aptamer, a small molecule, a polypeptide, a nucleic acid, a protein, or an antibody.

61. A pharmaceutical composition, comprising an aptamer of any one of claims 1 to 47.

62. A pharmaceutical composition, comprising an aptamer conjugate of any one of claims 48 to 61.

63. The pharmaceutical composition of claim 61 or 62, further comprising a pharmaceutically acceptable carrier.

64. The pharmaceutical composition of any one of claims 61 to 63, wherein the pharmaceutical composition is formulated for parenteral administration.

65. The pharmaceutical composition of any one of claims 61 to 64, for use in treating cancer.

66. The pharmaceutical composition of claim 65, wherein the cancer is a solid tumor.

67. The pharmaceutical composition of claim 66, wherein the cancer is a breast cancer, head and neck squamous cell carcinoma, adenoid cystic carcinoma, bladder cancer, pancreatic cancer, hepatocellular carcinoma, melanoma, merkel cell carcinoma, or a colorectal carcinoma.

68. A method of treating cancer, the method comprising administering to a subject an aptamer of any one of claims 1 to 47.

69. A method of treating cancer, the method comprising administering to a subject an aptamer conjugate of any one of claims 48 to 61.

70. A method of treating cancer, the method comprising administering to a subject a pharmaceutical composition of any one of claims 61 to 67.

71. The method of any one of claims 68 to 70, wherein the administration is parenteral administration.

72. The method of claim 71, wherein the administration is an intratumoral injection.

73. The method of any one of claims 68 to 72, wherein the cancer is a solid tumor.

74. The method of claim 73, wherein the cancer is a breast cancer, head and neck squamous cell carcinoma, adenoid cystic carcinoma, bladder cancer, pancreatic cancer, hepatocellular carcinoma, melanoma, merkel cell carcinoma, or a colorectal carcinoma.

75. The method of any one of claims 68 to 74, wherein the subject is a subject who has received chemotherapy.

76. The method of any one of claims 68 to 75, wherein the subject has had a tumor surgically removed.

77. The method of any one of claims 68 to 76, further comprising administering to the subject an additional cancer therapy.

78. The method of claim 77, wherein the additional cancer therapy comprises chemotherapy.

79. The method of claim 77, wherein the additional cancer therapy comprises radiation therapy.

80. The method of claim 77, wherein the additional cancer therapy comprises surgical removal of a tumor.

81. The method of claim 77, wherein the additional cancer therapy comprises administration of an immune checkpoint inhibitor to the subject.

82. The method of claim 81, wherein the immune checkpoint inhibitor is an anti-PD-1 antibody, an anti-PD-L1 antibody, an anti-PD-L2 antibody, or an anti-CTLA4 antibody.

83. A method of killing a cancer cell, the method comprising inducing CTL activity by contacting a CTL with an aptamer of any one of claims 1 to 47.

84. A method of killing a cancer cell, the method comprising contacting the cancer cell with an aptamer conjugate of any one of claims 48 to 61.

85. The method of claim 83 or 84, wherein the cancer cell is killed by apoptosis.

86. The method of any one of claims 83 to 85, wherein the cancer cell is a solid tumor cell.

87. The method of claim 86, wherein the cancer cell is a colorectal carcinoma cell.

88. The method of claim 86, wherein the cancer cell is a breast cancer cell.

89. A method of making an aptamer, the method comprising synthesizing a nucleic acid molecule comprising a sequence that is at least 80% identical to any one of SEQ ID NOs: 1-39, 59-77 or 80.

90. The method of claim 89, wherein the nucleic acid molecule comprises a nucleic acid sequence that is at least 90% identical to any one of SEQ ID NOs: 1-39, 59-77 or 80.

91. The method of claim 89 or 90, wherein the nucleic acid molecule comprises a nucleic acid sequence that is at least 95% identical to any one of SEQ ID NOs: 1-39, 59-77 or 80.

92. The method of any one of claims 89 to 91, wherein the nucleic acid molecule comprises a nucleic acid sequence that is at least 98% identical to any one of SEQ ID NOs: 1-39, 59-77 or 80.

93. The method of any one of claims 89 to 92, wherein the nucleic acid molecule comprises a nucleic acid sequence of any one of SEQ ID NOs: 1-39, 59-77 or 80.

94. The method of any one of claims 89 to 93, wherein the nucleic acid molecule comprises a nucleic acid sequence of any one of SEQ ID NOs: 3, 5, 6, 28, 29, 59 and 80.

95. A method of treating an autoimmune disorder in a subject comprising administering to the subject an aptamer of any one of claims 1-18 or 27-47.

96. A method of treating an inflammatory disease in a subject comprising administering to the subject an aptamer of any one of claims 1-18 or 27-47.

97. A method of inhibiting transplant rejection in a subject comprising administering to the subject an aptamer of any one of claims 1-18 or 27-47.

98. A method of treating an autoimmune disorder in a subject comprising administering to the subject a pharmaceutical composition of claim 61.

99. A method of treating an inflammatory disease in a subject comprising administering to the subject an aptamer of claim 61.

100. A method of inhibiting transplant rejection in a subject comprising administering to the subject an aptamer of claim 61.
